(19) United States
(12) Patent Application Publication
(10) Pub. No.: US 2025/0255916 A1
(43) Pub. Date: Aug. 14, 2025

(54) METHODS AND SYSTEMS FOR PROCESSING AND FORMULATING PLANT MATERIAL

(71) Applicant: FLOWERPOD, LLC, New Haven, CT (US)

(72) Inventors: Michael Fensterstock, New Haven, CT (US); Michael Sheldon, New Haven, CT (US)

(21) Appl. No.: 18/701,320

(22) PCT Filed: Oct. 14, 2022
(86) PCT No.: PCT/US2022/078167

§ 371 Date: Apr. 15, 2024

Related U.S. Application Data
(60) Provisional application No. 63/256,309, filed on Oct. 15, 2021.

(51) Int. Cl. A61K 36/185; B02B 1/02; B02B 1/08

(52) U.S. Cl. CPC A61K 36/3482; B02B 1/02; B02B 1/08; A61K 2236/15; A61K 2236/37; A61K 2236/51

(57) ABSTRACT

Disclosed herein is a method of modifying a plant material, a product produced by the method, and a system to implement the method.

### CROSS-REFERENCE

This application claims the benefit of U.S. Provisional Application No. 63/256,309, filed on Oct. 15, 2021, which is incorporated herein by reference in its entirety.

### BACKGROUND

Among the challenges associated with milling and/or formulating certain plant materials, for example Cannabis-derived flower material, by conventional means are issues associated with generating a flowable powder having a sufficiently small average particle size to be suitable for pressing and producing a pressed formulation suitable for vaping applications. The present disclosure relates to surprising innovations that render plant material, including sticky and/or viscous plant material such as Cannabis flower material, amenable to producing a flowable powder suitable for pressing, and having a sufficiently small particle size for use in vaping applications.

### SUMMARY

The present disclosure relates to methods and systems for modifying a plant material. In certain embodiments, the method of modifying a plant material comprises providing the plant material, wherein the plant material comprises at least 0.1% v/w of an oil content correlated to a fresh weight associated with a plant flower; milling the plant material at a first temperature to generate a first plurality of plant material fragments, wherein the first temperature is not in excess of +32 F, wherein the first plurality of plant material fragments comprises an average diameter of 300 μm and 1500 μm; and exposing the first plurality of plant material fragments to a second temperature not in excess of −25 F and a humidity not in excess of 40% RH for at least 1 hour. The present disclosure involves making and using

In an embodiment of the present disclosure, a method of modifying a plant material comprises providing the plant material, wherein the plant material comprises at least 0.1% v/w of an oil content correlated to a fresh weight associated with a plant flower; milling the plant material at a first temperature to generate a first plurality of plant material fragments, wherein the first temperature is not in excess of +32 F, wherein the first plurality of plant material fragments comprises an average diameter of 300-1500 μm; and exposing the first plurality of plant material fragments to a second temperature not in excess of +32 F and a humidity not in excess of 40% RH for at least 1 hour.

In some embodiments, exposing the first plurality of plant material fragments to a second temperature not in excess of +32 F and a humidity not in excess of 40% RH for at least 1 hour occurs produces a second plurality of plant material fragments. In some embodiments, the second plurality of plant material fragments comprise a flowable composition. In some embodiments, the plant material comprises at least 0.13% v/w oil content correlated to fresh weight associated with the plant flower. In some embodiments, the plant material comprises at least 0.16% v/w oil content correlated to fresh weight associated with the plant flower. In some embodiments, the plant material comprises at least 0.20% v/w oil content correlated to fresh weight associated with the plant flower. In some embodiments, the plant material comprises at least 0.29% v/w oil content correlated to fresh weight associated with the plant flower. In some embodiments, the oil content comprises a monoterpene profile or a sesquiterpene profile. In some embodiments, the monoterpene profile comprises between approximately 1% and 20% monoterpenes. In some embodiments, the sesquiterpene profile comprises between 1% and 20% sesquiterpenes. In some embodiments, the oil content comprises between 1% and 20% composition of monoterpenes. In some embodiments, the oil content comprises between 1% and 30% composition of sesquiterpenes. In some embodiments, the oil is a volatile oil or essential oil. In some embodiments, the plant material comprises between 1% and 30% v/w terpenes correlated to fresh weight associated with the plant flower. In some embodiments, the terpenes comprise a monoterpene profile or sesquiterpene profile. In some embodiments, the terpenes comprise between 1% and 30% monoterpenes. In some embodiments, the terpenes comprise between 1% and 30% sesquiterpenes. In some embodiments, the plant material comprises between 1% and 30% composition of monoterpenes. In some embodiments, the plant material comprises between 1% and 30% composition of sesquiterpenes. In some embodiments, the plant material comprises a cannabinoid profile. In some embodiments, the second temperature is the same as the first temperature. In some embodiments, exposing the first plurality of plant material fragments to a second temperature not in excess of +32 F and a humidity not in excess of 40% RH for at least 1 hour occurs after providing the plant material, wherein the plant material comprises at least 0.1% v/w of an oil content correlated to a fresh weight associated with a plant flower and before milling the plant material at a first temperature to generate a first plurality of plant material fragments, wherein the first temperature is not in excess of +32 F, wherein the first plurality of plant material fragments comprises an average diameter of 300-1500 μm. In some embodiments, exposing the first plurality of plant material fragments to a second temperature not in excess of +32 F and a humidity not in excess of 40% RH for at least 1 hour occurs after milling the plant material at a first temperature to generate a first plurality of plant material fragments, wherein the first temperature is not in excess of +32 F, wherein the first plurality of plant material fragments comprises an average diameter of 300-1500 μm. In some embodiments, the flowable composition comprises particles having an average diameter size between 300 and 1500 μm. In some embodiments, the method further comprises producing a formulation comprising the flowable composition. In some embodiments, producing the formulation further comprises compressing the flowable composition. In some embodiments, the compression occurs under a pressing force of up to 3 tons. In some embodiments, the compression occurs under a pressing force between approximately 0.5-1.7 tons. In some embodiments, the formulation comprises a puck or a tablet. In some embodiments, the puck or the tablet comprises a diameter between approximately 5 mm and 15 mm. In some embodiments, the puck or the tablet comprises a weight of between 1OOmg and 200 mg. In some embodiments, the puck or tablet comprises an aperture therein. In some embodiments, the compressing comprises maintaining the flowable composition at a storage temperature, a storage humidity, or a combination thereof, during the compression process. In some embodiments, the compressing further comprises reducing air moisture via an air dehumidifier. In some embodiments, the storage temperature is between approximately 30 F and 60 F. In some embodiments, the storage humidity is not more than 10% RH. In some embodiments, step a comprises introducing a coolant to an environment containing the plant material, wherein the coolant temperature is not in excess of +32 F. In some embodiments, the coolant comprises a solid, a gas, or a liquid. In some embodiments, the gas comprises nitrogen or carbon dioxide. In some embodiments, the milling in step b) comprises exposing the first plurality of plant material fragments to a first pressure, wherein the first pressure facilitates a shattering or a grinding of the plant material. In some embodiments, the first pressure is between 1000 and 14,000 kPa. In some embodiments, the second temperature is not in excess of −50 F. In some embodiments, the second temperature is between −50 F and +32 F. In some embodiments, exposing the first plurality of plant material fragments to a second temperature not in excess of +32 F and a humidity not in excess of 40% RH for at least 1 hour is performed for a duration between 1 hour and 16 hours. In some embodiments, exposing the first plurality of plant material fragments to a second temperature not in excess of +32 F and a humidity not in excess of 40% RH for at least 1 hour results in a reduction of a water content in the plant material by at least 90%. In some embodiments, exposing the first plurality of plant material fragments to a second temperature not in excess of +32 F and a humidity not in excess of 40% RH for at least 1 hour further comprises use of a Harvest Right® Freeze Dryers & At-Home Freeze Drying Accessories I Prep SOS™. In some embodiments, the first temperature in milling the plant material is modifiable based on a first ambient relative humidity. In some embodiments, the method further comprises reducing an ambient temperature of an ambient air, wherein the ambient air has a dew point temperature or a frost point temperature. In some embodiments, the dew point temperature or frost point temperature of the ambient air is lower than the first temperature, second temperature or storage temperature of the plant material. the dew point temperature is no less than +32 F. In some embodiments, the dew point temperature is between +32 F and +120 F. In some embodiments, the frost point temperature is not in excess of +32 F. In some embodiments, the storage temperature is modifiable based on a second ambient relative humidity during the compression process. In some embodiments, the second ambient relative humidity is the same as the first ambient relative humidity. In some embodiments, the method further comprises reducing air moisture via an air dehumidifier. In some embodiments, reducing air moisture via the air dehumidifier occurs at exposing the first plurality of plant material fragments to a second temperature. In some embodiments, the plant material comprises viscous components, sticky components, or a combination thereof. In some embodiments, the method further comprises introducing the plant material to a container configured to heat the plant material, the formulation, or the first or second plurality of plant material fragments to a heating temperature. In some embodiments, the heating temperature is no less than +315 F. In some embodiments, the heating temperature is between +315 F and +446 F.

In an embodiment, a product is produced by the methods disclosed herein. An embodiment of the present disclosure comprises a use of the product in an apparatus configured to heat the product. In some embodiments, the apparatus comprises a vape, an electronic pipe, or device configured to heat the product.

In an embodiment, a use of the product is for medicinal or recreational purposes.

Another embodiment of the present disclosure comprises a system for producing a flowable particle comprising a temperature-modifying apparatus configured to alter a temperature of a plant material or a plurality of fragments thereof, wherein the plant material comprises at least 0.1% v/w of an oil content correlated to a fresh weight associated with a plant flower, a milling device configured to process the plant material and produce a plurality of plant material fragments, a dehumidifier to alter a moisture content of the plurality of plant material fragments or an ambient relative humidity, and a compression device configured to compress the plant material or the plurality of fragments thereof, wherein the compression produces a formulation. In some embodiments, the temperature-modifying apparatus comprises a series of channels configured to transport a coolant, wherein the series of channels are located in the milling device and the compression device. In some embodiments, the coolant comprises a liquid or a gas. In some embodiments, the milling device further comprises a ball mill. In some embodiments, the ball mill is a hard plastic, a metal, or a combination thereof. In some embodiments, the ball mill is operated for a length of time that achieves a powdered flower material having a desired average particle size to be suitable for producing the formulation. In some embodiments, the desired average particle size is between 300 μm and 1500 μm. In some embodiments, the length of time comprises 1 minute to 30 minutes. In some embodiments the milling device comprises a tumbling machine comprising a grinder configured to ground the plant material to an average particle size suitable for producing the formulation. In some embodiments, the average particle size suitable for producing the formulation is between 300 μm and 1500 μm. In some embodiments, the milling device further comprises an Original Resonator OG CBD Kief Separator, H20 Extraction and Cryo-Trimt_{1M} Machine. In some embodiments, the milling device rotates at a rate of 15 RPM to 150 RPM. In some embodiments, the plant material comprises viscous components, sticky components or a combination thereof. In some embodiments, the formulation comprises a puck or a tablet. In some embodiments, the compression device is configured to maintain or modify a storage temperature, a storage humidity, or a combination thereof, of the plant material or plurality of fragments thereof during the compression. In some embodiments, the oil is an essential oil or a volatile oil.

In another embodiment, a method of modifying a plant material comprises providing the plant material, wherein the plant material comprises at most 20% w/w of terpenes correlated to fresh weight, milling the plant material at a first temperature to generate a first plurality of plant material fragments, wherein the first temperature is not in excess of +32 F, wherein the first plurality of plant material fragments comprises an average diameter between approximately 300 μm and 1500 μm, and exposing the first plurality of plant material fragments to a second temperature not in excess of −25 F and a humidity not in excess of 10% RH for at least 1 hour.

In some embodiments, exposing the first plurality of plant material fragments to a second temperature not in excess of −25 F and a humidity not in excess of 10% RH for at least 1 hour occurs after providing the plant material, wherein the plant material comprises at most 20% w/w of terpenes correlated to fresh weight and before milling the plant material at a first temperature to generate a first plurality of plant material fragments, wherein the first temperature is not in excess of +32 F, wherein the first plurality of plant material fragments comprises an average diameter between approximately 300 μm and 1500 μm.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings.

FIGS. 1A-B depict a solid puck IOO (FIG. 1A) and a donut-shaped or bagel-shaped puck IOI (FIG. 1B). In some cases, the donut-shaped or bagel-shaped puck comprises a puck having an aperture therein. In both Figures IA and IB, two coins (a U.S. 25 cent coin, larger, and a U.S. IO cent coin, smaller) and a AAA format battery are shown for scale.

FIGS. 2A-B depict methods of modifying a plant material of the present disclosure. In some cases, exposing the plant material to a temperature can include freeze drying the plant material. In some cases, freeze drying the plant material occurs at a temperature not more than +32 F. In some cases, freeze drying the plant material occurs at a temperature not more than OF. In some cases, the steps listed in FIG. 2A and FIG. 2B are executed in a system as disclosed herein. The system can include a series of components, wherein the series of components comprises a temperature-modifying apparatus, a milling device, a dehumidifier, and a compression device. In some cases, the temperature-modifying apparatus is in direct communication with the milling device, the dehumidifier, and the compression device. In some cases, the temperature-modifying apparatus is in direct contact with the milling device, the dehumidifier, and the compression device. In some cases, the temperature-modifying apparatus comprises a series of channels

FIGS. 3A-D depict components of a system disclosed herein. In some cases, the system can include a tablet press machine and parts therein. FIG. 3A depicts an insert, lower tool configured to produce a puck having a diameter of 7.8 mm and a press assembly. FIG. 3B depicts an insert, upper tool configured to produce a puck having a diameter of 7.8 mm and a press assembly FIG. 3C depicts an insert, upper tool configured to produce a puck having a diameter of 8.3 mm and a press assembly. FIG. 3D depicts an insert, lower tool configured to produce a puck having a diameter of 8.3 mm and a press assembly. FIGS. 3A-D depict geometric tolerance and dimension tolerance, including alignment specifications, fraction specifications, angle specification, and sizing measurements of the tablet press machine and parts therein providing accurate sizing, circumferences, and depth of fill. In some embodiments, a user can control lower and upper tools or press components to reach a desired level of compress10n.

FIGS. 4A-B depict components of a system disclosed herein. In some cases, the system can include a tablet press machine and parts therein FIG. 4A depicts a die cavity, two bore, tooling assembly and press assembly for producing a puck disclosed herein, wherein the puck produced has a diameter of approximately 8.3 mm. FIG. 4B depicts a die, two bore, tooling assembly and press assembly configured to produce a 7.8 mm puck as disclosed herein. FIGS. 4A-B depict geometric tolerance and dimension tolerance, including alignment specifications, fraction specifications, angle specification, and sizing measurements of the tablet press machine and parts therein providing accurate sizing, circumferences, and depth of fill. In some embodiments, a user can control lower and upper tools or press components to reach a desired level of compression. In some embodiments, compression occurs in a compression chamber.

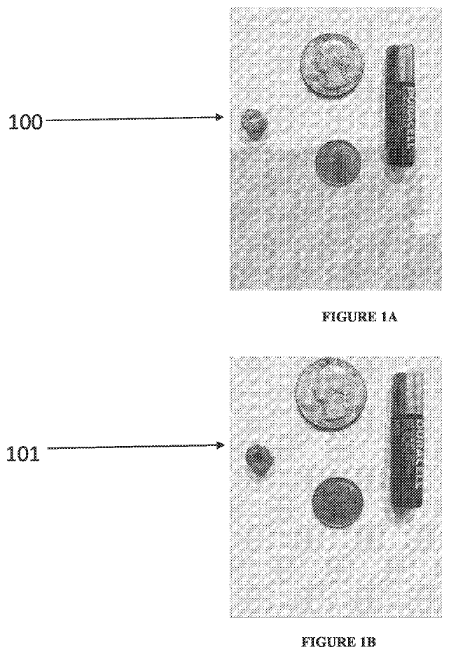

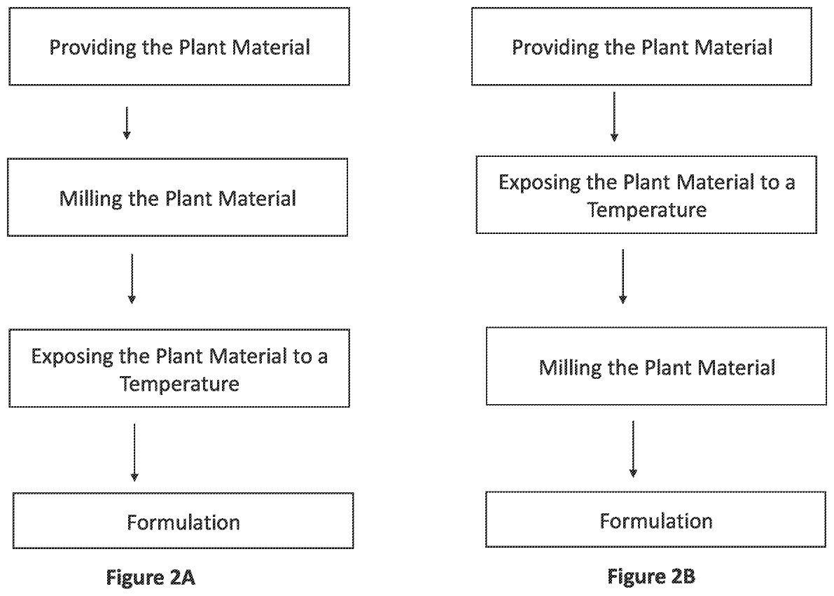

### DETAILED DESCRIPTION

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the disclosure can also be implemented in a single embodiment.

Reference in the specification to “some embodiments,” “an embodiment,” “one embodiment” or “other embodiments” means that a feature, structure, or characteristic described in connection with the embodiments is included in at least some embodiments, but not necessarily all embodiments, of the present disclosure.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the disclosure can be used to achieve methods of the disclosure.

The term “about” or “approximately” as used herein when referring to a measurable value such as a parameter, an amount, a temporal duration, and the like, is meant to encompass variations of +/−30% or less, +/−20% or less, +/−10% or less, +/−5% or less, or +/−1% or less of and from the specified value, insofar such variations are appropriate to perform in the present disclosure. It is to be understood that the value to which the modifier “about” or “approximately” refers is itself also specifically disclosed.

Embodiments of the present disclosure relate to the surprising discovery that by controlling one or more parameters of the milling and/or formulating process, plant material that is not suitable for milling and/or formulation through pressing. In embodiments disclosed herein, methods and systems which control certain parameters in the milling and pressing processes, including without limitation temperature and humidity, result in advantages over conventions methods and systems. In some embodiments, the plant material and/or components of the systems of the present disclosure are chilled, and/or maintained in a reduced humidity environment, during the milling and/or pressing process.

### Starter Plant Material

In some embodiments, the plant material can have viscous components, sticky components or a combination thereof. In some embodiments, the plant material can be a Cannabis plant material. In some embodiments, the Cannabis plant material comprises Cannabis sativa, Cannabis indica, Cannabis ruderalis, or a hybrid combination thereof. In some embodiments, the plant material comprises a hemp plant material. In some embodiments, the plant material can be derived from a plant in its flowering stage. In some embodiments, the plant material can be derived from a plant in a harvest or curing stage.

In some embodiments, the plant material can be an orgamc plant material. In some embodiments, the organic plant material can include flower, cola, bracts, trichomes, node, fan leaves, sugar leaves, stem, pistils, and stigmas. In some embodiments, trichomes can be sticky glands that cover the flowers and small leaves of the Cannabis plant. In some embodiments, trichomes can house the plants cannabinoids and terpenes. In some embodiments, trichomes can be bulbous trichomes, capitate-sessile trichomes or capitate-stalked trichomes. In some embodiments, the components of the Cannabis plant material flower can include a pistil and stigma, cola, bract and calyx. In some embodiments, the flower comprises a bud or a marijuana bud.

In some embodiments, the plant material's life cycle can be in germination for 3-10 days, seedling for 2-3 weeks, vegetative for 3-16 weeks and flowering for 8-11 weeks.

In some embodiments, the plant material can have a full-spectrum profile of an origin plant. In some embodiments, the plant material can have a partial-spectrum profile with less than a full-spectrum profile of the origin plant. In some embodiments, the full-spectrum profile of the plant material comprises all chemical constituents of an origin plant. In some embodiments, the full-spectrum profile of the plant material comprises all the chemical constituents of the origin plant which produce a therapeutic effect. In some embodiments, the origin plant is the plant which created the plant material. In some embodiments, the origin plant is Cannabis sativa, Cannabis indica, Cannabis ruderalis, or a hybrid combination thereof. In some embodiments, the plants Cannabis sativa, Cannabis indica, Cannabis ruderalis, or a hybrid combination thereof are collectively referred to as “the Cannabis plant, the Cannabis plant material, or the Cannabis.” In some embodiments, the full-spectrum profile of the plant material comprises all the chemical constituents of the Cannabis plant which produce the therapeutic effect. In some embodiments, the chemical constituents of the Cannabis plant which produce the therapeutic effect include cannabinoids and terpenes.

In some embodiments, the origin plant material can have a cannabinoid profile. In some embodiments, cannabinoids can be a group of substances found in the Cannabis plant. In some embodiments, the full-spectrum profile of the Cannabis plant can have up to 113 cannabinoids.

In some embodiments, the full-spectrum profile comprises a variety of cannabinoids, including Tetrahydrocannabinol (THC), Tetrahydrocannabinol acid (THCa), Cannabidiol (CBD), Cannabidiolic acid (CBDa), Cannabigerol (CBG), and Cannabinol (CBN). In some embodiments, the full-spectrum profile comprises delta-9-tetrahydrocannabinol. In some embodiments, the tetrahydrocannabinol (THC) is the principal psychoactive constituent of Cannabis. In some embodiments, the CBD is derived from hemp. In some embodiments, the plant material with the partial-spectrum profile of the origin plant material comprises all the chemical constituents of the Cannabis plant which produce the therapeutic effect. In some embodiments, the plant material with the partial-spectrum profile comprises a variety of cannabinoids, including Tetrahydrocannabinol (THC), Tetrahydrocannabinol acid (THCa), Cannabidiol (CBD), Cannabidiolic acid (CBDa), Cannabigerol (CBG), and Cannabinol (CBN). In some embodiments, the partial-spectrum profile comprises delta-9-tetrahydrocannabinol. In some cases, the full-spectrum profile comprises flavonoids, proteins, phenols, sterols, and esters. In some cases, the partial-spectrum profile comprises flavonoids, proteins, phenols, sterols, and esters.

In some cases, the full-spectrum or partial-spectrum profile comprises terpenes. In some embodiments, the full-spectrum or partial-spectrum profile of the plant material comprises all the terpenes of the origin plant. In some embodiments, the full-spectrum or partial-spectrum profile of the plant material comprises all of the monoterpenes, sesquiterpenes, diterpenes and triterpenes of the origin plant material.

In some embodiments, the terpenes can be hydrocarbon chemicals. In some embodiments, terpenes can be the primary constituents of essential oils or volatile oils. In some embodiments, terpenes can be responsible for the aroma characteristics of the plant material. In some embodiments, terpenes can be monoterpenes, sesquiterpenes, diterpenes, or triterpenes. In some embodiments, the Cannabis plant can include over 400 terpenes.

In some cases, the plant material can have a natural terpene content. In some embodiments, the natural terpene content is anywhere between approximately 1% and 10%. In some embodiments, terpenes can be added (i.e., exogenous terpenes) to the first plurality of plant material fragments or to the second plurality of plant material fragments. In some cases, the product produced after adding exogenous terpenes can have a terpene content of up to 40%. In some cases, the product produced after adding exogenous terpenes can have a terpene content of more than 10%.

In some embodiments, terpenes produce a therapeutic effect by interreacting with the endocannabinoid system.

In some embodiments, the plant material can have a monoterpene profile. In some embodiments, the Cannabis plant material can have a monoterpene profile. In some embodiments, the monoterpene profile can be any of a class of terpenes containing two isoprene units per molecule. In some embodiments, the monoterpene can have the chemical formula C10H16. In some embodiments, the monoterpene profile can have myrcene, limonene, -myrcene, a-pinene, and linalool, a-terpinolene or tran-ocimene, or a combination thereof. In some embodiments, the plant material can have up to 58 monoterpenes. In some embodiments, tobacco plant material can have a monoterpene profile.

In some embodiments, the plant material can have a sesquiterpene profile. In some embodiments, the Cannabis plant material can have a sesquiterpene profile. In some embodiments, the sesquiterpene profile can be any of a class of terpenes containing three isoprene units per molecule. In some embodiments, the sesquiterpene can have the chemical formula C1sH24 In some embodiments, the sesquiterpene profile can have -caryophyllene. In some embodiments, the sesquiterpene profile can have a terpene known to bind to the cannabinoid receptor CB2. In some embodiments, the sesquiterpene profile can have a E-caryophyllene, caryophyllene oxide, E-famesene, and -caryophyllene. In some embodiments, the sesquiterpene profile can have up to 38 sesquiterpenes. In some embodiments, tobacco plant material can have a sesquiterpene profile.

In some embodiments, the plant material disclosed herein can have a terpene concentration or content between approximately 1% and 15%. In some embodiments, the formulation, puck or tablet disclosed herein can have a terpene concentration or content between approximately 1% and 30%. In some embodiments, the plant material disclosed herein can have a monoterpene concentration or content between approximately 1% and 15%. In some embodiments, the formulation, puck or tablet disclosed herein can have a monoterpene concentration or content between approximately 1% and 30%. In some embodiments, the plant material disclosed herein can have a sesquiterpene concentration or content between approximately 1% and 15%. In some embodiments, the formulation, puck or tablet disclosed herein can have a sesquiterpene concentration or content between approximately 1% and 30%.

In some embodiments, the plant material can have a diterpene profile. In some embodiments, the diterpene profile can be any of a class of terpenes containing four isoprene units per molecule. In some embodiments, tobacco plant material can have a diterpene profile. In some embodiments, the Cannabis plant material can have a diterpene profile. In some embodiments, the diterpene can have the chemical formula C20H32.

In some embodiments, the plant material can have a triterpene profile. In some embodiments, the triterpene profile can be any of a class of terpenes containing six isoprene units per molecule. In some embodiments, tobacco plant material can have a triterpene profile. In some embodiments, the Cannabis plant material can have a triterpene profile. In some embodiments, the triterpene can have the chemical formula C3oH4s.

In some embodiments, the plant material can have a full spectrum terpene profile. In some embodiments, the full spectrum terpene profile comprises all the terpenes of the original plant. In some embodiments, the full spectrum terpene profile comprises all the monoterpenes, sesquiterpenes, diterpenes, triterpenes or a combination thereof of the original plant.

In some embodiments, the plant material can be a Cannabis plant material. In some embodiments, the Cannabis plant material comprises a THC-dominant strain. In some embodiments, the THC-dominant strain can be a flower with a high concentration of THC, CBD and other cannabinoids. In some embodiments, the plant material flower can comprise a percent composition of THC selected from the following percentages: 0%, between 0% and 11%, between 1% and 2%, between 2% and 3%, between 3% and 4%, between 4% and 5%, between 5% and 6%, between 6% and 7%, between 7% and 8%, between 8% and 9%, between 9% and 10%, between 10% and 11%, between 11% and 12%, between 12% and 13%, between 13% and 14%, between 14% and 15%, between 15% and 16%, between 16% and 17%, between 17% and 18%, between 18% and 19%, between 19% and 20%, between 20% and 21%, between 21% and 22%, between 22% and 23%, between 23% and 24%, between 24% and 25%, between 25% and 26%, between 26% and 27%, between 27% and 28%, between 28% and 29%, between 29% and 30%, between 30% and 31%, between 31% and 32%, between 32% and 33%, between 33% and 34%, between 34% and 35%, and 35%.

In some embodiments, the plant material is a CBD-dominant strain. In some embodiments, the plant material can be a flower with a high concentration of CBD, THC, other cannabinoids, or a combination thereof. In some embodiments, the plant material flower can comprise a percent composition of CBD selected from the following percentages: 0%, between 0% and 1%, between 1% and 2%, between 2% and 3%, between 3% and 4%, between 4% and 5%, between 5% and 6%, between 6% and 7%, between 7% and 8%, between 8% and 9%, between 9% and 10%, between 10% and 11%, between 11% and 12%, between 12% and 13%, between 13% and 14%, between 14% and 15%, between 15% and 16%, between 16% and 17%, between 17% and 18%, between 18% and 19%, between 19% and 20%, between 20% and 21%, between 21% and 22%, between 22% and 23%, between 23% and 24%, between 24% and 25%, between 25% and 26%, between 26% and 27%, between 27% and 28%, between 28% and 29%, between 29% and 30%, and 30%.

In some embodiments, a Cannabis resin can be a substance found on the trichomes of the Cannabis plant. In some embodiments, the Cannabis resin can contain high concentration levels of THC. In some embodiments, high concentration levels of THC can comprise a percent composition of THC selected from the following percentages: 10%, between 10% and 11%, between 11% and 12%, between 12% and 13%, between 13% and 14%, between 14% and 15%, between 15% and 16%, between 16% and 17%, between 17% and 18%, between 18% and 19%, between 19% and 20%, between 20% and 21%, between 21% and 22%, between 22% and 23%, between 23% and 24%, between 24% and 25%, between 25% and 26%, between 26% and 27%, between 27% and 28%, between 28% and 29%, between 29% and 30%, between 30% and 31%, between 31% and 32%, between 32% and 33%, between 33% and 34%, between 34% and 35%, and 35%. In some embodiments, the Cannabis resin can be hash, butane hash oil, live resin, rosin, or C02 oil.

In some embodiments, the method comprises producing the Cannabis resin. In some embodiments, the Cannabis resin can be a live resin also referred to as a live resin concentrate. In some embodiments, the live resin concentrate can be a Cannabis concentrate extracted from freshly harvested Cannabis plant. In some embodiments, live resin is a Cannabis concentrate extracted from freshly frozen Cannabis plant flower. In some embodiments, live resin can be extracted from fresh frozen flower which is Cannabis flower that is frozen immediately after harvest to preserve freshness until the extraction process. In some embodiments, live resin can be extracted from Cannabis buds or Cannabis plant material using solvents. In some embodiments, the solvents can be light hydrocarbon solvents. In some embodiments, the solvents can be butane, propane, alcohol or carbon dioxide. In some embodiments, live resin can be decarboxylated. In some embodiments, live resin can have a consistency between looser concentrates like sauce and distillate and more solid extracts like wax and rosin. In some embodiments, live resin can be thicker and chunky with a crystally texture. In some embodiments, live resin can be used in vape cartridges. In some embodiments, live resin can be used on its own. In some embodiments, live resin can be in edibles where it is decarboxylated. In some embodiments, the THC content of live resin can be greater than 90% depending on the plant material. In some embodiments, the THC content of live resin can be between 50%-90%. In some embodiments, the live resin can be CBD live resin. In some embodiments, the CBD live resin can have a CBD content between 1% and 90%. In some embodiments, the live resin can have a strong flavor and aroma due to its high terpene content. In some embodiments, the terpene content of live resin can be between 1% and 40%.

In some embodiments, the method comprises producing a distillate. In some embodiments, the distillate can be a Cannabis distillate. In some embodiments, the Cannabis distillate can have a low terpene content. In some embodiments, the terpene content is not in excess of 5%. In some embodiments, the Cannabis distillate can have high potency and versatility. In some embodiments, the Cannabis distillate comprises up to 98% cannabinoids. In some embodiments, undistilled extracts comprise approximately 60-80% cannabinoids. In some embodiments, the Cannabis distillate can be produced by a series of extraction and purification processes, removing undesirable plant matter, resulting in an ultra-refined, nearly pure Cannabis extract with up to 98% Cannabinoids. In some embodiments, the Cannabis distillate can be extracted with butane, propane, alcohol or carbon dioxide. In some embodiments, the Cannabis distillate can be vaporized, dabbed, smoked, eaten, or topically applied. In some embodiments, the Cannabis distillate can be used in vape cartridges, edibles, and topicals. In some embodiments, the Cannabis distillate can be in syringes, which can be used to dab, eat, or refill cartridges.

In some embodiments, the Cannabis live resin and the Cannabis distillate can be vaporized, dabbed, inhaled, ingested, or smoked, and can be used in edibles, topicals, and other products.

In some embodiments, “v/w” can be the volume of oil divided by the fresh weight of plant material. In some embodiments, the fresh weight of the plant material can be the weight of the freshly collected buds. In some embodiments, the volume of the oil can be associated with plant material that has been dried. In some embodiments, the volume of the oil can be associated with plant material that has not been dried. In some embodiments, the volume of the oil can be associated with freshly collected buds that have been dried. In some embodiments, the volume of the oil can be associated with freshly collected buds that have not been dried.

In some embodiments, the oil of the plant material can be volatile oil or essential oil. In some embodiments, the oil of the plant material can have a viscosity that is dependent on the temperature and composition of the oil. In some embodiments, the oil can be a dark viscous liquid. In some embodiments, the viscosity of the oil having a composition between [78%-80]% THC and [0%-0.29%]% CBD and temperature between [40-70]° C. can be between [169.9 cP-5666]cP. In some embodiments, the oil is Cannabis flower essential oil, also known as hemp essential oil. In some embodiments, the Cannabis plant material essential oil is obtained by steam distillation from the flowers, panicles (flower cluster), stem, and upper leaves of the hemp plant.

In some embodiments, Cannabis extracts can be a broad range of products that generally contain higher levels of cannabinoids than are found in the Cannabis plant.

In some embodiments, Cannabis extracts can be in solid form (hash or hashish) or in liquid form (oil intended for vaping). In some embodiments, extracts are named after their appearance or consistency, such as shatter, wax, honeycomb and budder. In some embodiments, Cannabis extracts are typically smoked or vaporized, and some can be added to dried Cannabis flower.

In some embodiments, the plant material is flower or other organic material. In some embodiments, the material is derived from a tobacco plant. In some embodiments, the plant material comprises viscous components, sticky components, or a combination thereof, making conventional methods for processing plant material, for example by milling, difficult or impossible. In some embodiments, the plant or flower material is derived from a CBD dominant or a THC dominant Cannabis plant, or from any other Cannabis plant with or without full spectrum terpene profiles.

In some embodiments of the present disclosure, the plant material, flower material, or a combination thereof is derived from a Cannabis spp. In some embodiments, the flower material is derived from Cannabis sativa plants, Cannabis indica plants, Cannabis ruderalis plants, or a hybrid combination thereof.

In some embodiments, the plant material is processed into a flowable material, such as a powder. In some embodiments, the flowable material or powder is then formulated into a solid form, such as a puck or tablet. In some embodiments, the resulting puck or tablet is suitable for smoking or vaping applications.

In some embodiments, the plant material comprises a cannabinoid profile. In some embodiments, the plant material comprises over 100 cannabinoids. In some embodiments, the plant material comprises over 113 cannabinoids.

In some embodiments, the plant material comprises Tetrahydrocannabinol (THC). In some embodiments, the plant material comprises cannabidiol (CBD).

In some embodiments, the plant material comprises terpenes. In some embodiments, the plant material comprises monoterpenes. In some embodiments, the plant material comprises not in excess of 58 monoterpenes. In some embodiments, the monoterpenes comprise limonene, -myrcene, a-pinene, linalool, a-terpinolene, tran-ocimene or a combination thereof.

In some embodiments, the plant material comprises sesquiterpenes. In some embodiments, the plant material comprises not in excess of 38 sesquiterpenes. In some embodiments, the sesquiterpenes comprises -caryophyllene, the only terpene known to bind to the cannabinoid receptor CB2; E-caryophyllene, caryophyllene oxide, E-famesene, -caryophyllene or a combination thereof.

In some embodiments, the plant material has an oil content. In some embodiments, the oil is in the flowers or buds of the plant material. In some embodiments, the oil content is a volatile oil or essential oil. In some embodiments, the flower can have an oil content selected from the following range: 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, and 0.30% volume of oil content correlated to fresh weight of the flowers or buds of the plant material.

In some embodiments, the oil content of the flower of the plant material has terpenes. In some embodiments, the oil content of the flower of the plant material comprises monoterpenes. In some embodiments, the oil content of the flower of the plant material comprises not in excess of 58 monoterpenes. In some embodiments, the monoterpenes comprise limonene, -myrcene, a-pinene, linalool, a-terpinolene or tran-ocimene. In some embodiments, the oil content of the flower of the plant material comprises a percent composition of monoterpenes selected from the following range: 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%.monoterpenes. In some embodiments, the oil content of the flower of the plant material comprises between 62%-93% monoterpenes.

In some embodiments, the oil content of the flower of the plant material comprises sesquiterpenes. In some embodiments, the oil content of the flower of the plant material comprises not in excess of 38 sesquiterpenes. In some embodiments, the sesquiterpenes comprises -caryophyllene, the only terpene known to bind to the cannabinoid receptor CB2; E-caryophyllene, caryophyllene oxide, E-famesene, or -caryophyllene. In some embodiments, the oil content of the flower of the plant material comprises a percent composition of sesquiterpenes selected from the following range: 0%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%. In some embodiments, the oil content of the flower of the plant material comprises between 6%-35% sesquiterpenes.

In some embodiments, the plant material can be a tobacco plant material. In some embodiments, the tobacco plant material can be a naturally occurring tobacco plant material. In some embodiments, the plant material can be a tobacco-cannabis hybrid plant material. In some embodiments, the plant material can be cannabis combined with any naturally occurring plant material. In some embodiments, the tobacco plant material can be Nicotiana tabacum. In some embodiments, the plant material can be Aztec tobacco (N. rustica). In some embodiments, the plant material can be a cured leaf In some embodiment, the cured leaf is aged and processed. In some embodiments, the cured leaf can be comprised in a composition for a use. In some embodiments, the use comprises smoking, chewing, snuffing, and extraction of nicotine. In some embodiments, the starting plant material can be used to produce a composition for use in a smoking product, a chewing product, a snuffing product, and an extraction of nicotine product. In some embodiments, the plant material can be a species of the genus Nicotiana that are grown as ornamentals, known collectively as flowering tobaccos.

### Milling

In some embodiments, milling can be grinding flower material to a first plurality of plant material fragments with a preferred micron size range. In some embodiments, the preferred micron size can be 300 μm-1500 μm.

In some embodiments, a flowable composition can be a plant material in powder form comprising the first plurality of plant material fragments.

In some embodiments, the flowable composition can have a sufficiently small average particle size to be suitable for pressing and producing a pressed formulation. In some embodiments, a flowable composition can have sufficiently small particle size for use in smoking applications. In some embodiments, a flowable composition can have sufficiently small particle size for use in vaping applications. In some embodiments, a flowable composition can have sufficiently small particle size for use in an electronic pipe. In some embodiments, a flowable composition can comprise particles with an average diameter between 300 μm-1500 μm.

In some embodiments, powder flowability refers to the ability of a powder to flow in a desired manner in a specific piece of equipment.

In some embodiments, the flowable compositions flowability and powder flowability can be measured by density indices, such as the Carr index and Hausner ratio, powder avalanching, the angle of repose (AOR), flow through an orifice, powder rheometry and shear cell testing.

In some embodiments, powder flow can be influenced by environmental factors, such as moisture and static electricity, as well as powder related factors, such as morphology, size, size distribution, density, and surface area.

In some embodiments, a first temperature can be the temperature of the plant material during the milling process. In some embodiments, the first temperature can be the temperature of the plant material prior to milling.

In some embodiments, a first ambient relative humidity can be a humidity of the air or environment surrounding the system during milling. In some embodiments, the first ambient relative humidity can be a humidity of the air or environment surrounding the system during freeze drying. In some embodiments, the first ambient relative humidity can be a percent relative humidity (% RH) between 30% RH to 90% RH. In some embodiments, the first ambient relative humidity during milling is a percent relative humidity (% RH) selected from the following range: I %, 2%, 3%, 4%, S %, 6%, 7%, 8%, 9%, IO %, 11%, I2%, 13%, I4%, IS %, I6%, I7%, I8%, I9%, 20%, 2I %, 22%, 23%, 24%, 2S %, 26%, 27%, 28%, 29%, 30%, 3I %, 32%, 33%, 34%, 3S %, 36%, 37%, 38%, 39%, 40%, 4I %, 42%, 43%, 44%, 4S %, 46%, 47%, 48%, 49%, SO %, SI %, S2%, S3%, S4%, SS %, S6%, S7%, S8%, S9%, 60%, 6I %, 62%, 63%, 64%, 6S %, 66%, 67%, 68%, 69%, 70%, 7I %, 72%, 73%, 74%, 7S %, 76%, 77%, 78%, 79%, 80%, 8I %, 82%, 83%, 84%, 8S %, 86%, 87%, 88%, 89%, 90%, 9I %, 92%, 93%, 94%, 9S %, 96%, 97%, 98%, and 99%.

In some embodiments, the air moisture can be the content of water vapor in the air or environment surrounding the system.

In some embodiments, an ambient air temperature can be the temperature of the ambient air or environment surrounding the system during the method described herein. In some embodiments, the ambient air temperature can be selected from the following range: −99 F, −98 F, −97 F, −96 F, −9SF, −94 F, −93 F, −92 F, −9IF, −90 F, −89 F, −88 F, −87 F, −86 F, −8SF, −84 F, −83 F, −82 F, −8IF, −80 F, −79 F, −78 F, −77 F, −76 F, −7SF, −74 F, −73 F, −72 F, −7IF, −70 F, −69 F, −68 F, −67 F, −66 F, −6SF, −64 F, −63 F, −62 F, −6IF, −60 F, −S9 F, −S8 F, −S7 F, −S6 F, −SSF, −S4 F, −S3 F, −S2 F, −SIF, −SOF, −49 F, −48 F, −47 F, −46 F, −4SF, −44 F, −43 F, −42 F, −4IF, −40 F, −39 F, −38 F, −37 F, −36 F, −3SF, −34 F, −33 F, −32 F, −3IF, −30 F, −29 F, −28 F, −27 F, −26 F, −2SF, −24 F, −23 F, −22 F, −2IF, −20 F, −I9 F, −I8 F, −I7 F, −I6 F, −ISF, −I4 F, −13 F, −I2 F, −I IF, −IOF, −9 F, −8 F, −7 F, −6 F, −SF, −4 F, −3 F, −2 F, −IF, OF, IF, 2 F, 3 F, 4 F, SF, 6 F, 7 F, 8 F, 9 F, IOF, IIF, I2 F, 13 F, I4 F, ISF, 16 F, 17 F, 18 F, 19 F, 20 F, 2IF, 22 F, 23 F, 24 F, 2SF, 26 F, 27 F, 28 F, 29 F, 30 F, 3IF, 32 F, 33 F, 34 F, 3SF, 36 F, 37 F, 38 F, 39 F, 40 F, 4IF, 42 F, 43 F, 44 F, 4SF, 46 F, 47 F, 48 F, 49 F, SOF, SIF, S2 F, S3 F, S4 F, SSF, S6 F, S7 F, S8 F, S9 F, 60 F, 6IF, 62 F, 63 F, 64 F, 6SF, 66 F, 67 F, 68 F, 69 F, 70 F, 7IF, 72 F, 73 F, 74 F, 7SF, 76 F, 77 F, 78 F, 79 F, 80 F, 81 F, 82 F, 83 F, 84 F, 8SF, 86 F, 87 F, 88 F, 89 F, 90 F, 91 F, 92 F, 93 F, 94 F, 9SF, 96 F, 97 F, 98 F, and 99 F.

In some embodiments, a frost point temperature can be the temperature where at certain conditions including ambient relative humidity water vapor in the air will condense as a layer of frost onto systems of the present disclosure. In some embodiments, frost point temperature can be a temperature not in excess of +32 F. In some embodiments, the frost point temperature can be selected from the following range: −99 F, −98 F, −97 F, −96 F, −9SF, −94 F, −93 F, −92 F, −91 F, −90 F, −89 F, −88 F, −87 F, −86 F, −8SF, −84 F, −83 F, −82 F, −81 F, −80 F, −79 F, −78 F, −77 F, −76 F, −7SF, −74 F, −73 F, −72 F, −71 F, −70 F, −69 F, −68 F, −67 F, −66 F, −6SF, −64 F, −63 F, −62 F, −61 F, −60 F, −S9 F, −S8 F, −S7 F, −S6 F, −SSF, −S4 F, −S3 F, −S2 F, −SIF, −SOF, −49 F, −48 F, −47 F, −46 F, −4SF, −44 F, −43 F, −42 F, −41 F, −40 F, −39 F, −38 F, −37 F, −36 F, −3SF, −34 F, −33 F, −32 F, −3IF, −30 F, −29 F, −28 F, −27 F, −26 F, −2SF, −24 F, −23 F, −22 F, −21 F, −20 F, −19 F, −18 F, −17 F, −16 F, −ISF, −14 F, −13 F, −12 F, −11 F, −IOF, −9 F, −8 F, −7 F, −6 F, −SF, −4 F, −3 F, −2 F, −IF, OF, IF, 2 F, 3 F, 4 F, SF, 6 F, 7 F, 8 F, 9 F, IOF, 11 F, 12 F, 13 F, 14 F, ISF, 16 F, 17 F, 18 F, 19 F, 20 F, 21 F, 22 F, 23 F, 24 F, 2SF, 26 F, 27 F, 28 F, 29 F, 30 F, 31 F, 32 F, 33 F, 34 F, 3SF, 36 F, 37 F, 38 F, 39 F, 40 F, 41 F, 42 F, 43 F, 44 F, 4SF, 46 F, 47 F, 48 F, 49 F, SOF, SIF, S2 F, S3 F, S4 F, SSF, S6 F, S7 F, S8 F, S9 F, 60 F, 61 F, 62 F, 63 F, 64 F, 6SF, 66 F, 67 F, 68 F, 69 F, 70 F, 71 F, 72 F, 73 F, 74 F, 7SF, 76 F, 77 F, 78 F, 79 F, 80 F, 81 F, 82 F, 83 F, 84 F, 8SF, 86 F, 87 F, 88 F, 89 F, 90 F, 91 F, 92 F, 93 F, 94 F, 9SF, 96 F, 97 F, 98 F, and 99 F.

In some embodiments, a device, system, apparatus, or a combination thereof removes water vapor or a water content from the air. In some embodiments, an air dehumidifier can be the device, system, apparatus for removing water vapor from the air.

In some embodiments, a coolant can be a material which lowers the temperature of the plant material. In some embodiments, the coolant is also referred to as a chilling material. In some embodiments, the coolant can be dry ice. In some embodiments, the coolant can be a chilling fluid. In some embodiments, the chilling fluid can be a gas (nitrogen or carbon dioxide) or liquid that results in cold temperatures of the plant material. In some embodiments, the coolant renders the plant or flower material subject to shattering when struck by ball mills. In some embodiments, the chilling fluid is nitrogen, carbon dioxide, or a combination thereof. In some embodiments, the temperature of the coolant can be between −SOF and +32 F. In some embodiments, the temperature of the coolant can be selected from the following range: −99 F, −98 F, −97 F, −96 F, −9SF, −94 F, −93 F, −92 F, −91 F, −90 F, −89 F, −88 F, −87 F, −86 F, −8SF, −84 F, −83 F, −82 F, −81 F, −80 F, −79 F, −78 F, −77 F, −76 F, −7SF, −74 F, −73 F, −72 F, −71 F, −70 F, −69 F, −68 F, −67 F, −66 F, −6SF, −64 F, −63 F, −62 F, −61 F, −60 F, −S9 F, −S8 F, −S7 F, −S6 F, −SSF, −S4 F, −S3 F, −S2 F, −SIF, −SOF, −49 F, −48 F, −47 F, −46 F, −4SF, −44 F, −43 F, −42 F, −41 F, −40 F, −39 F, −38 F, −37 F, −36 F, −3SF, −34 F, −33 F, −32 F, −3IF, −30 F, −29 F, −28 F, −27 F, −26 F, −2SF, −24 F, −23 F, −22 F, −21 F, −20 F, −19 F, −18 F, −17 F, −16 F, −ISF, −14 F, −13 F, −12 F, −11 F, −IOF, −9 F, −8 F, −7 F, −6 F, −SF, −4 F, −3 F, −2 F, −IF, OF, IF, 2 F, 3 F, 4 F, SF, 6 F, 7 F, 8 F, 9 F, IOF, 11 F, 12 F, 13 F, 14 F, I SF, 16 F, 17 F, 18 F, 19 F, 20 F, 21 F, 22 F, 23 F, 24 F, 2SF, 26 F, 27 F, 28 F, 29 F, 30 F, 31 F, 32 F, 33 F, 34 F, 3SF, 36 F, 37 F, 38 F, 39 F, 40 F, 41 F, 42 F, 43 F, 44 F, 4SF, 46 F, 47 F, 48 F, 49 F, SOF, SIF, S2 F, S3 F, S4 F, SSF, S6 F, S7 F, S8 F, S9 F, 60 F, 61 F, 62 F, 63 F, 64 F, 6SF, 66 F, 67 F, 68 F, 69 F, 70 F, 71 F, 72 F, 73 F, 74 F, 7SF, 76 F, 77 F, 78 F, 79 F, 80 F, 81 F, 82 F, 83 F, 84 F, 8SF, 86 F, 87 F, 88 F, 89 F, 90 F, 91 F, 92 F, 93 F, 94 F, 9SF, 96 F, 97 F, 98 F, and 99 F. In some embodiments, the temperature of the plant material, after being subjected to the coolant, can be selected from the following range: −99 F, −98 F, −97 F, −96 F, −9SF, −94 F, −93 F, −92 F, −91 F, −90 F, −89 F, −88 F, −87 F, −86 F, −8SF, −84 F, −83 F, −82 F, −81 F, −80 F, −79 F, −78 F, −77 F, −76 F, −7SF, −74 F, −73 F, −72 F, −71 F, −70 F, −69 F, −68 F, −67 F, −66 F, −6SF, −64 F, −63 F, −62 F, −61 F, −60 F, −S9 F, −S8 F, −S7 F, −S6 F, −SSF, −S4 F, −S3 F, −S2 F, −SIF, −SOF, −49 F, −48 F, −47 F, −46 F, −4SF, −44 F, −43 F, −42 F, −41 F, −40 F, −39 F, −38 F, −37 F, −36 F, −3SF, −34 F, −33 F, −32 F, −3IF, −30 F, −29 F, −28 F, −27 F, −26 F, −2SF, −24 F, −23 F, −22 F, −21 F, −20 F, −19 F, −18 F, −17 F, −16 F, −ISF, −14 F, −13 F, −12 F, −11 F, −IOF, −9 F, −8 F, −7 F, −6 F, −SF, −4 F, −3 F, −2 F, −1 F, OF, 1 F, 2 F, 3 F, 4 F, SF, 6 F, 7 F, 8 F, 9 F, IOF, 11 F, 12 F, 13 F, 14 F, ISF, 16 F, 17 F, 18 F, 19 F, 20 F, 21 F, 22 F, 23 F, 24 F, 2SF, 26 F, 27 F, 28 F, 29 F, 30 F, 31 F, 32 F, 33 F, 34 F, 3SF, 36 F, 37 F, 38 F, 39 F, 40 F, 41 F, 42 F, 43 F, 44 F, 4SF, 46 F, 47 F, 48 F, 49 F, SOF, SlF, S2 F, S3 F, S4 F, SSF, S6 F, S7 F, S8 F, S9 F, 60 F, 61 F, 62 F, 63 F, 64 F, 6SF, 66 F, 67 F, 68 F, 69 F, 70 F, 71 F, 72 F, 73 F, 74 F, 7SF, 76 F, 77 F, 78 F, 79 F, 80 F, 81 F, 82 F, 83 F, 84 F, 8SF, 86 F, 87 F, 88 F, 89 F, 90 F, 91 F, 92 F, 93 F, 94 F, 9SF, 96 F, 97 F, 98 F, and 99 F.

In some embodiments, a milling device or apparatus can be a system for grounding the flower material to a preferred micron size range. In some embodiments, the milling device or apparatus can crush the plant material into extremely fine form. In some embodiments, the milling can comprise up milling. In some embodiments, the milling can comprise down milling. In some embodiments, the particle can be an average size between approximately 300 μm and ISOO μm. In some embodiments, the particle can be an average diameter between approximately 300 μm and ISOO μm. In some embodiments, the ball mill can be made of hard plastic, a metal, or a combination thereof. In some embodiments, the ball mill can be made of stainless steel or rubber. In some embodiments, the milling device can be a tumbling machine. In some embodiments, balls can vary in size, from small marbles to about the size of golf balls.

In some embodiments, the material is milled in a milling machine, which may be a ball mill. In some embodiments, milling machines are configured to spin or rotate either clockwise or counterclockwise. In some embodiments, the milling machines can rotate between 15 RPM-120 RPM during the milling process.

In some embodiments, the milling process employs the Original Resinator 0(3 CBD Kief Separator, H20 Extraction and Cryo-Trinfrm Machine The Original Resinator htips://www.theoriginalresinator.comiproductiresi nator-og-extracti on-mach in et according to instructions for use, the contents of which are incorporated by reference in their entirety. In some embodiments, the ball mill is operated for 5 minutes to 25 minutes to achieve powdered flower material having a desired average particle size or size range. In some embodiments, the ball mill is operated for a number of minutes to achieve powdered flower material having a desired average particle size or size range selected from the following range: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, and 99 minutes.

In some embodiments, pre-roll ground means the flower is ready for grinding. In some embodiments, the flower can be pre-roll ground. In some embodiments, the flower material can be ground by a grinder. In some embodiments, the grinder breaks up the plant material into plurality of plant material particles with a preferred micron size. In some embodiments, the preferred micron size can comprise particles with an average size between approximately 300 μm and 1500 μm.

In some embodiments, the material produced by milling can be flowable and suitable for processing by pressing into tablets or pucks. In some embodiments, the material can be maintained at cooled temperatures, reduced humidity, or a combination thereof, which may improve maintenance in a flowable state prior to pressing. In some embodiments, the material may be processed by freeze drying, as discussed further below.

In some embodiments, the milling can comprise exposing the first plurality of plant material fragments to a first pressure, wherein the first pressure facilitates a shattering or a grinding of the plant material. In some embodiments, the first pressure is between 1000 and 6000 kPa.

In some embodiments, the temperature of the plant material during milling is modified based on the first ambient relative humidity. In some embodiments, the temperature of the plant material during milling is selected from the following range: −99 F, −98 F, −97 F, −96 F, −95 F, −94 F, −93 F, −92 F, −91 F, −90 F, −89 F, −88 F, −87 F, −86 F, −85 F, −84 F, −83 F, −82 F, −81 F, −80 F, −79 F, −78 F, −77 F, −76 F, −75 F, −74 F, −73 F, −72 F, −71 F, −70 F, −69 F, −68 F, −67 F, −66 F, −65 F, −64 F, −63 F, −62 F, −61 F, −60 F, −59 F, −58 F, −57 F, −56 F, −55 F, −54 F, −53 F, −52 F, −51 F, −50 F, −49 F, −48 F, −47 F, −46 F, −45 F, −44 F, −43 F, −42 F, −41 F, −40 F, −39 F, −38 F, −37 F, −36 F, −35 F, −34 F, −33 F, −32 F, −31 F, −30 F, −29 F, −28 F, −27 F, −26 F, −25 F, −24 F, −23 F, −22 F, −21 F, −20 F, −19 F, −18 F, −17 F, −16 F, −15 F, −14 F, −13 F, −12 F, −11 F, −10 F, −9 F, −8 F, −7 F, −6 F, −5 F, −4 F, −3 F, −2 F, −1 F, OF, 1 F, 2 F, 3 F, 4 F, 5 F, 6 F, 7 F, 8 F, 9 F, 1OF, 11 F, 12 F, 13 F, 14 F, 15 F, 16 F, 17 F, 18 F, 19 F, 20 F, 21 F, 22 F, 23 F, 24 F, 25 F, 26 F, 27 F, 28 F, 29 F, 30 F, 31 F, 32 F, 33 F, 34 F, 35 F, 36 F, 37 F, 38 F, 39 F, 40 F, 41 F, 42 F, 43 F, 44 F, 45 F, 46 F, 47 F, 48 F, 49 F, 50 F, 51 F, 52 F, 53 F, 54 F, 55 F, 56 F, 57 F, 58 F, 59 F, 60 F, 61 F, 62 F, 63 F, 64 F, 65 F, 66 F, 67 F, 68 F, 69 F, 70 F, 71 F, 72 F, 73 F, 74 F, 75 F, 76 F, 77 F, 78 F, 79 F, 80 F, 81 F, 82 F, 83 F, 84 F, 85 F, 86 F, 87 F, 88 F, 89 F, 90 F, 91 F, 92 F, 93 F, 94 F, 95 F, 96 F, 97 F, 98 F, 99 F. In some embodiments, the ambient relative humidity during milling is a percent selected from the following range: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%.

In some embodiments, the ambient air has an ambient air temperature. In some embodiments, the ambient air temperature is between approximately 20 F and 99 F. In some embodiments, the ambient air temperature can be: 20 F, 21 F, 22 F, 23 F, 24 F, 25 F, 26 F, 27 F, 28 F, 29 F, 30 F, 31 F, 32 F, 33 F, 34 F, 35 F, 36 F, 37 F, 38 F, 39 F, 40 F, 41 F, 42 F, 43 F, 44 F, 45 F, 46 F, 47 F, 48 F, 49 F, 50 F, 51 F, 52 F, 53 F, 54 F, 55 F, 56 F, 57 F, 58 F, 59 F, 60 F, 61 F, 62 F, 63 F, 64 F, 65 F, 66 F, 67 F, 68 F, 69 F, 70 F, 71 F, 72 F, 73 F, 74 F, 75 F, 76 F, 77 F, 78 F, 79 F, 80 F, 81 F, 82 F, 83 F, 84 F, 85 F, 86 F, 87 F, 88 F, 89 F, 90 F, 91 F, 92 F, 93 F, 94 F, 95 F, 96 F, 97 F, 98 F, or 99 F.

In some embodiments, the ambient air temperature has a dew point temperature. In some embodiments, the dew point temperature is no less than +32 F. In some embodiments, the dew point temperature is between +32 F and +120 F. In some embodiments, the dew point temperature is selected from the following range: 32 F, 33 F, 34 F, 35 F, 36 F, 37 F, 38 F, 39 F, 40 F, 41 F, 42 F, 43 F, 44 F, 45 F, 46 F, 47 F, 48 F, 49 F, 50 F, 51 F, 52 F, 53 F, 54 F, 55 F, 56 F, 57 F, 58 F, 59 F, 60 F, 61 F, 62 F, 63 F, 64 F, 65 F, 66 F, 67 F, 68 F, 69 F, 70 F, 71 F, 72 F, 73 F, 74 F, 75 F, 76 F, 77 F, 78 F, 79 F, 80 F, 81 F, 82 F, 83 F, 84 F, 85 F, 86 F, 87 F, 88 F, 89 F, 90 F, 91 F, 92 F, 93 F, 94 F, 95 F, 96 F, 97 F, 98 F, 99 F, 1OOF, 1O1 F, 102 F, 103 F, 104 F, 105 F, 106 F, 107 F, 108 F, 109 F, 11OF, 111 F, 112 F, 113 F, 114 F, 115 F, 116 F, 117 F, 118 F, 119 F, 120 F. In some embodiments, the ambient air temperature has a frost point temperature not in excess of +32 F. In some embodiments, the frost point temperature can be −50, −49, −48, −47, −46, −45, −44, −43, −42, −41, −40, −39, −38, −37, −36, −3S, −34, −33, −32, −31, −30, −29, −28, −27, −26, −2 S, −24, −23, −22, −21, −20, −19, −18, −17, −16, −IS, −14, −13, −12, −11, −10, −9, −8, −7, −6, −S, −4, −3, −2, −1, 0, 1, 2, 3, 4, S, 6, 7, 8, 9, 10, 11, 12, 13, 14, IS, 16, 17, 18, 19, 20, 21, 22, 23, 24, 2S, 26, 27, 28, 29, 30, 31, or 32 F.

In some embodiments, the ambient air temperature is reduced. In some embodiments, reduction of the ambient air temperature results in the ambient air having a reduced dew point temperature. In some embodiment, the temperature of the plant material is increased to avoid condensation onto the plant material. In some embodiments, the ambient air temperature is increased. In some embodiments, the ambient air temperature is increased to increase the dew point temperature of the ambient air. In some embodiments, increasing the dew point temperature of the ambient air results in reduced condensation onto the plant material.

### Methods—Grinding

In some embodiments, the flower is ground. In some embodiments, the flower is received flower from cultivator and placed in a −3S+degree freezer for at least 2 hours. In some embodiments, the freezer is at a temperature of 99 F, −98 F, −97 F, −96 F, −9SF, −94 F, −93 F, −92 F, −91 F, −90 F, −89 F, −88 F, −87 F, −86 F, −8SF, −84 F, −83 F, −82 F, −81 F, −80 F, −79 F, −78 F, −77 F, −76 F, −7SF, −74 F, −73 F, −72 F, −71 F, −70 F, −69 F, −68 F, −67 F, −66 F, −6SF, −64 F, −63 F, −62 F, −61 F, −60 F, −S9 F, −S8 F, −S7 F, −S6 F, −SSF, −S4 F, −S3 F, −S2 F, −SIF, −SOF, −49 F, −48 F, −47 F, −46 F, −4SF, −44 F, −43 F, −42 F, −41 F, −40 F, −39 F, −38 F, −37 F, −36 F, −3SF, −34 F, −33 F, −32 F, −3IF, −30 F, −29 F, −28 F, −27 F, −26 F, −2SF, −24 F, −23 F, −22 F, −21 F, −20 F, −19 F, −18 F, −17 F, −16 F, −ISF, −14 F, −13 F, −12 F, −11 F, −IOF, −9 F, −8 F, −7 F, −6 F, −SF, −4 F, −3 F, −2 F, −IF, OF, IF, 2 F, 3 F, 4 F, SF, 6 F, 7 F, 8 F, 9 F, IOF, 11 F, 12 F, 13 F, 14 F, ISF, 16 F, 17 F, 18 F, 19 F, 20 F, 21 F, 22 F, 23 F, 24 F, 2SF, 26 F, 27 F, 28 F, 29 F, 30 F, 3IF, and 32 F. In some embodiments, the grinder is inspected. In some embodiments, it is assured that the grinder is fully cleaned or, if not, it is cleaned. In some embodiments, the grinder is cleaned. In some embodiments, the grinder is in contact with a coolant. In some embodiments, the coolant is capable of cleaning the grinder. In some embodiments, the mesh, grinding chamber and blades are disassembled. In some embodiments, the grinder is cleaned with a 3-bay sink: soap & water, rinse, and sanitizer. In some embodiments, the grinder is dried and reassembled. In some embodiments the flower is preroll ground. In some embodiments, the grinder comprises an 840 micron mesh.

In some embodiments, the flower is not ground. In some embodiments, the preroll mesh is inserted into the grinder. In some embodiments, the flower is ground with a preroll mesh. After the flower is ground to that bigger size, the flower is refreezed for approximately two hours. Following refreezing, the preroll mesh is replaced with a clean 840 micron mesh and move to the next step. In some embodiments, once the flower is “preroll ground”, the frozen flower is ground in the HUUE grinder with 840 micron mesh. In some embodiments, a flow is checked periodically. A low grind flow is low can be due to an uncleaned or a clogged mesh. In some cases, the 840 micron mesh is very fine, requiring for the material to be cold to process without gumming up the mesh. In some cases, the mesh is scraped to improve flow or possibly remove and clean the mesh. In some cases, once ground, the flower is combined with the concentrate formula and blend. In some embodiments, either manually mix the material in a bowl at least SO times until fully blended or use a clean tumbler to combine. In some embodiments, place the ground and mixed material in the freeze dryer if it is being compressed the next day, or return material to the freezer. In some embodiments, the grinder is cleaned. In some embodiments, the mesh, grinding chamber and blades are disassembled. In some embodiments, the grinder is cleaned with 3 bay sinks: soap & water, rinse, and sanitizer. In some embodiments, the grinder is dried and reassembled.

### Temperatures

In some embodiments, a temperature-modifying apparatus can be an apparatus configured to the alter the temperature of the plant material, the first plurality of fragments of the plant material, the second plurality of fragments of the plant material, another plurality of fragments of the plant material, or a combination thereof. In some embodiments, the temperature-modifying apparatus can alter the temperature of the plant material to a first temperature. In some embodiments, the temperature-modifying apparatus can alter the temperature of the plant material to a second temperature.

In some embodiments, the temperature-modifying apparatus can be the freeze dryer. In some embodiments, the temperature-modifying apparatus can be a Harvest Right® Freeze Dryers & At-Home Freeze Drying Accessories I Prep SOS™. In some embodiments, the freeze dryer can alter the temperature of the plant material to a temperature selected from the following range: −99 F, −98 F, −97 F, −96 F, −9SF, −94 F, −93 F, −92 F, −9IF, −90 F, −89 F, −88 F, −87 F, −86 F, −8SF, −84 F, −83 F, −82 F, −8IF, −80 F, −79 F, −78 F, −77 F, −76 F, −7SF, −74 F, −73 F, −72 F, −7IF, −70 F, −69 F, −68 F, −67 F, −66 F, −6SF, −64 F, −63 F, −62 F, −6IF, −60 F, −S9 F, −SSF, −S7 F, −S6 F, −SSF, −S4 F, −S3 F, −S2 F, −SIF, −SOF, −49 F, −48 F, −47 F, −46 F, −4SF, −44 F, −43 F, −42 F, −4IF, −40 F, −39 F, −38 F, −37 F, −36 F, −3SF, −34 F, −33 F, −32 F, −3IF, −30 F, −29 F, −28 F, −27 F, −26 F, −2SF, −24 F, −23 F, −22 F, −2IF, −20 F, −I9 F, −18 F, −I7 F, −I6 F, −ISF, −14 F, −I3 F, −I2 F, −IIF, −IOF, −9 F, −8 F, −7 F, −6 F, −SF, −4 F, −3 F, −2 F, −IF, OF, IF, 2 F, 3 F, 4 F, SF, 6 F, 7 F, 8 F, 9 F, IOF, IIF, I2 F, I3 F, I4 F, ISF, 16 F, 17 F, 18 F, 19 F, 20 F, 2IF, 22 F, 23 F, 24 F, 2SF, 26 F, 27 F, 28 F, 29 F, 30 F, 3IF, 32 F, 33 F, 34 F, 3SF, 36 F, 37 F, 38 F, 39 F, 40 F, 4IF, 42 F, 43 F, 44 F, 4SF, 46 F, 47 F, 48 F, 49 F, SOF, SIF, S2 F, S3 F, S4 F, SSF, S6 F, S7 F, S8 F, S9 F, 60 F, 6IF, 62 F, 63 F, 64 F, 6SF, 66 F, 67 F, 68 F, 69 F, 70 F, 7IF, 72 F, 73 F, 74 F, 7SF, 76 F, 77 F, 78 F, 79 F, 80 F, 8IF, 82 F, 83 F, 84 F, 8SF, 86 F, 87 F, 88 F, 89 F, 90 F, 9IF, 92 F, 93 F, 94 F, 9SF, 96 F, 97 F, 98 F, and 99 F.

In some embodiments, the temperature modifying apparatus can be the coolant or chilling material. In some embodiments, the coolant or a chilling material can be a material which lowers the temperature of the plant material. In some embodiments, the temperature of the plant material is not more than +32 F. In some embodiments, the coolant can be dry ice. In some embodiments, the coolant can be a chilling fluid. In some embodiments, the chilling fluid can be a gas. In some embodiments, the chilling fluid can be nitrogen, carbon dioxide, or a combination thereof. In some embodiments, the chilling fluid is a liquid that results in the plant or flower material with a temperature cold enough to render the plant or flower material subject to shattering when struck by ball mills. In some embodiments, the temperature of the coolant can be between −SOF and +32 F. In some embodiments, the temperature of the coolant can be selected from the following range: −99 F, −98 F, −97 F, −96 F, −9SF, −94 F, −93 F, −92 F, −9IF, −90 F, −89 F, −88 F, −87 F, −86 F, −8SF, −84 F, −83 F, −82 F, −8IF, −80 F, −79 F, −78 F, −77 F, −76 F, −7SF, −74 F, −73 F, −72 F, −7IF, −70 F, −69 F, −68 F, −67 F, −66 F, −6SF, −64 F, −63 F, −62 F, −6IF, −60 F, −S9 F, −S8 F, −S7 F, −S6 F, −SSF, −S4 F, −S3 F, −S2 F, −SIF, −SOF, −49 F, −48 F, −47 F, −46 F, −4SF, −44 F, −43 F, −42 F, −4IF, −40 F, −39 F, −38 F, −37 F, −36 F, −3SF, −34 F, −33 F, −32 F, −3IF, −30 F, −29 F, −28 F, −27 F, −26 F, −2SF, −24 F, −23 F, −22 F, −2IF, −20 F, −I9 F, −I8 F, −I7 F, −I6 F, −ISF, −I4 F, −I3 F, −I2 F, −IIF, −IOF, −9 F, −8 F, −7 F, −6 F, −SF, −4 F, −3 F, −2 F, −IF, OF, IF, 2 F, 3 F, 4 F, SF, 6 F, 7 F, 8 F, 9 F, IOF, IIF, I2 F, 13 F, I4 F, ISF, 16 F, 17 F, 18 F, 19 F, 20 F, 2IF, 22 F, 23 F, 24 F, 2SF, 26 F, 27 F, 28 F, 29 F, 30 F, 3IF, 32 F, 33 F, 34 F, 3SF, 36 F, 37 F, 38 F, 39 F, 40 F, 4IF, 42 F, 43 F, 44 F, 4SF, 46 F, 47 F, 48 F, 49 F, SOF, SIF, S2 F, S3 F, S4 F, SSF, S6 F, S7 F, S8 F, S9 F, 60 F, 6IF, 62 F, 63 F, 64 F, 6SF, 66 F, 67 F, 68 F, 69 F, 70 F, 7IF, 72 F, 73 F, 74 F, 7SF, 76 F, 77 F, 78 F, 79 F, 80 F, 8IF, 82 F, 83 F, 84 F, 8SF, 86 F, 87 F, 88 F, 89 F, 90 F, 9IF, 92 F, 93 F, 94 F, 9SF, 96 F, 97 F, 98 F, and 99 F.

In some embodiments, the second temperature can be the temperature of the plant material after the milling process. In some embodiments, the second temperature can be the temperature of the plant material prior to milling. In some embodiments, the second temperature can be the temperature of the plant material during milling. In some embodiments, the second temperature can be the temperature of the plant material during freeze drying.

In some embodiments, freeze drying the plant material comprises placing the plant material into a freeze dryer. In some embodiments, the freeze dryer is configured to have a temperature of no more than −1OF. In some cases, C02 is not injected into the freeze dryer. In some embodiments, plant material can be a stale plant material.

In some embodiments, the second temperature can be the temperature of the plant material prior to compression. In some embodiments, the second temperature can be selected from the following range: −99 F, −98 F, −97 F, −96 F, −9SF, −94 F, −93 F, −92 F, −91 F, −90 F, −89 F, −88 F, −87 F, −86 F, −8SF, −84 F, −83 F, −82 F, −81 F, −80 F, −79 F, −78 F, −77 F, −76 F, −7SF, −74 F, −73 F, −72 F, −71 F, −70 F, −69 F, −68 F, −67 F, −66 F, −6SF, −64 F, −63 F, −62 F, −61 F, −60 F, −S9 F, −S8 F, −S7 F, −S6 F, −SSF, −S4 F, −S3 F, −S2 F, −SIF, −SOF, −49 F, −48 F, −47 F, −46 F, −4SF, −44 F, −43 F, −42 F, −41 F, −40 F, −39 F, −38 F, −37 F, −36 F, −3SF, −34 F, −33 F, −32 F, −3IF, −30 F, −29 F, −28 F, −27 F, −26 F, −2SF, −24 F, −23 F, −22 F, −21 F, −20 F, −19 F, −18 F, −17 F, −16 F, −ISF, −14 F, −13 F, −12 F, −11 F, −IOF, −9 F, −8 F, −7 F, −6 F, −SF, −4 F, −3 F, −2 F, −IF, OF, IF, 2 F, 3 F, 4 F, SF, 6 F, 7 F, 8 F, 9 F, IOF, IIF, 12 F, 13 F, 14 F, ISF, 16 F, 17 F, 18 F, 19 F, 20 F, 21 F, 22 F, 23 F, 24 F, 2SF, 26 F, 27 F, 28 F, 29 F, 30 F, 31 F, 32 F, 33 F, 34 F, 3SF, 36 F, 37 F, 38 F, 39 F, 40 F, 41 F, 42 F, 43 F, 44 F, 4SF, 46 F, 47 F, 48 F, 49 F, SOF, SIF, S2 F, S3 F, S4 F, SSF, S6 F, S7 F, S8 F, S9 F, 60 F, 61 F, 62 F, 63 F, 64 F, 6SF, 66 F, 67 F, 68 F, 69 F, 70 F, 71 F, 72 F, 73 F, 74 F, 7SF, 76 F, 77 F, 78 F, 79 F, 80 F, 81 F, 82 F, 83 F, 84 F, 8SF, 86 F, 87 F, 88 F, 89 F, 90 F, 91 F, 92 F, 93 F, 94 F, 9SF, 96 F, 97 F, 98 F, and 99 F. In some embodiments, the second temperature of the plant material can be less than, greater than, or equal to the first temperature.

In some embodiments, the second temperature is modified based upon the first ambient relative humidity (% RH), second ambient relative humidity, or both and is a % RH selected from the following range: 1%, 2%, 3%, 4%, S %, 6%, 7%, 8%, 9%, 10%, 11%, 12%, 13%, 14%, IS %, 16%, 17%, 18%, 19%, 20%, 21%, 22%, 23%, 24%, 2 S %, 26%, 27%, 28%, 29%, 30%, 31%, 32%, 33%, 34%, 3 S %, 36%, 37%, 38%, 39%, 40%, 41%, 42%, 43%, 44%, 4 S %, 46%, 47%, 48%, 49%, SO %, SI %, S2%, S3%, S4%, SS %, S6%, S7%, S8%, S9%, 60%, 61%, 62%, 63%, 64%, 6S %, 66%, 67%, 68%, 69%, 70%, 71%, 72%, 73%, 74%, 7S %, 76%, 77%, 78%, 79%, 80%, 81%, 82%, 83%, 84%, 8S %, 86%, 87%, 88%, 89%, 90%, 91%, 92%, 93%, 94%, 9S %, 96%, 97%, 98%, and 99%.

In some embodiments, the plant material is provided. In some embodiments, the coolant or chilling material is introduced to the plant material. In some embodiments, the coolant or chilling material is introduced prior to milling. In some embodiments, the plant material maintains the coolant or chilling material temperature during milling. In some embodiments, the plant material maintains the coolant or chilling material temperature during the entire method of the present disclosure. In some embodiments, the coolant comprises a solid. In some embodiments, the chilling material is dry ice. In some embodiments, the chilling material is a chilling fluid. In some embodiments, the chilling fluid comprises a gas or liquid. In some embodiments, the chilling gas comprises nitrogen or carbon dioxide. In some embodiments, the introduction of chilling material results in sufficiently cold temperatures to render the plant or flower material subject to shattering when struck by ball mills.

In some embodiments, the chilling material imparts a temperature of between −50 F to +32 F to the plant material or the flower material. In some embodiments, the chilling material cools the flower material or the plant material to a temperature between approximately −50 F and +32 F. In some embodiments, the flower material is from a Cannabis plant. In some embodiments, the flower material can be a tobacco plant material. In some embodiments, the chilling material imparts a sufficiently cold temperature on the plant or flower material to facilitate shattering, grinding, or a combination thereof of the material down to particles having an average desired micron size or size range. In some embodiments, the desired micron or size range is between approximately 300 μm and 1500 μm.

In some embodiments, exposing the first plurality of plant material fragments to a second temperature not in excess of +60 F and a humidity not in excess of 40% RH for at least 1 hour produces a second plurality of plant material fragments. In some embodiments, the plant material is exposed to a second pressure prior to or after milling. In some embodiments, the second pressure is not in excess of 800 mtorr (15.5 PSI). In some embodiments, the plant material is exposed to the second temperature in a vacuum. In some embodiments, the second plurality of plant material fragments comprises a flowable composition. In some embodiments, the second temperature of the plant material is the same as the first temperature of the plant material.

In some embodiments, exposing the first plurality of plant material fragments to a second temperature not in excess of +60 F and a humidity not in excess of 40% RH for at least 1 hour produces a second plurality of plant material fragments occurs prior to milling. In some embodiments, exposing the first plurality of plant material fragments to a second temperature not in excess of +60 F and a humidity not in excess of 40% RH for at least 1 hour produces a second plurality of plant material fragments occurs after milling. In some embodiments, exposing the first plurality of plant material fragments to a second temperature not in excess of +60 F and a humidity not in excess of 40% RH for approximately 12 hours produces a second plurality of plant material fragments.

In some embodiments, the second temperature is between −50 F to +60 F. In some embodiments, the second temperature is not in excess of −25 F. In some embodiments, the second temperature is maintained constant. In some embodiments, the second temperature fluctuates between −50 F to +60 F.

In some embodiments, exposing the first plurality of plant material fragments to a second temperature not in excess of +60 F and a humidity not in excess of 40% RH for between 1 hour and 16 hours. In some embodiments, exposing the first plurality of plant material fragments to a second temperature not in excess of 0 F and a humidity not in excess of 40% RH for between 1 hour and 16 hours results in a reduction of a water content in the plant material by at least 95%. In some embodiments, exposing the first plurality of plant material fragments to a second temperature not in excess of +60 F and a humidity not in excess of 40% RH for between 1 hour and 16 hours. In some embodiments, exposing the first plurality of plant material fragments to a second temperature not in excess of +60 F F and a humidity not in excess of 40% RH for between 1 hour and 16 hours results the second plurality of plant material fragments comprises between approximately 0% and 5% water.

In some embodiments, exposing the first plurality of plant material fragments to a second temperature not in excess of 0 F and a humidity not in excess of 40% RH for between 1 hour and 16 hours results in a reduction of a water content in the plant material by a percent selected from the following range: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%. In some embodiments, exposing the first plurality of plant material fragments to a second temperature not in excess of 0 F and a humidity not in excess of 40% RH for between 1 hour and 16 hours proceeds with the use of Harvest Right Freeze Dryers Freeze Dryers & At-Home Freeze Drying Accessories Prep SOS and according to the recommended methods for use set out in the accompanying materials and/or manuals, the contents of which are incorporated by reference in their entirety.

### Methods—Freeze Drying SOP

In some embodiments, it is assured that the freeze dryer is fully cleaned or, if not, it is cleaned. In some embodiments the freeze drier is cleaned. In some embodiments, the freeze dryer is cleaned by using water, paper towels, and no alcohol. In some embodiments, clean the plexiglass and rubber to assure a good future vacuum, and wipe off any material on the trays. In some embodiments, once the trays are emptied, clean with 3 bay sink: soap & water, rinse, sanitizer. In some embodiments, place the trays back in the freeze dryer. In some embodiments, it is assured that the drain hose is inside a 5-gallon bucket. In some embodiments, it is assured that the freeze dryer settings are 60 F degrees maximum temperature and close/seal the plexiglass front door. In some embodiments, press start and after approximately 15 minutes it will cool and follow instruction on the screen. In some embodiments, it is assured that a drain valve is closed on left side near rear of unit. In some embodiments, load material onto trays and place trays in the freeze dryer. In some embodiments, press continue once. In some embodiments, the freeze dryer will run for approximately 12 hours. In some embodiments, the freeze dryer will automatically stop and maintain internal temperature of approximately −35 degrees. In some embodiments, open the drain valve above any water in the bucket to eliminate the chamber vacuum.

In some embodiments, the plant material is observed for wanted or unwanted physical properties. In some embodiments, if the plant material is dry and fluffy, it is ready for compression (i.e., wanted). In some embodiments, if the plant material still slightly wet, return to the chamber, close drain valve, and press “more dry time” (i.e., unwanted). In some embodiments, the freeze dryer will run for approximately 2 hours more. In some embodiments, once all materials have been removed, press defrost and let the system run for approximately 2 hours. In some embodiments, the freeze drier is cleaned. In some embodiments, the freeze dryer is cleaned by using water, paper towels, and no alcohol. In some embodiments, clean the plexiglass and rubber to assure a good future vacuum, and wipe off any material on the trays. In some embodiments, once the trays are emptied, clean with 3 bay sink: soap & water, rinse, sanitizer. In some embodiments, place the trays back in the freeze dryer. In some embodiments, it is assured that the drain hose is inside a 5-gallon bucket. In some embodiments, the freeze dryer is set to a temperature of no more than OF and the room in which the freeze dryer is contained is set to a temperature no more than 60 F, and the freeze dryer is closed/sealed via a plexiglass front door.

### Formulation

In some embodiments, the powder material derived from the milling process described above can be formulated by pressing also known as compressing into pucks or tablets, which may be suitable for vaping or smoking applications. In some embodiments, the puck or tablet can comprise an aperture therein resembling a donut or bagel. In some embodiments, the circumference of the puck or tablet and vertical length of the puck or tablet can make them suitable for vaping applications, smoking applications, or a combination thereof.

In some embodiments, the puck or tablet comprises a mass can be O. 1 g, 0.2 g, 0.3 g, 0.4 g, 0.5 g, 0.6 g, 0.7 g, 0.8 g, 0.9 g, or 1.Og.

In some embodiments, a storage temperature can be a temperature of the plant material, flowable composition, or flowable powder during the compression process. In some embodiments, the storage temperature can be 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 2s° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., 100° C., 101° C., 102° C., 103° C., 104° C., 105° C., 106° C., 107° C., 108° C., 109° C., 110° C., 111° C., 112° C., 113° C., 114° C., 115° C., 116° C., 117° C., 118° C., 119° C., 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C., 141° C., 142° C., 143° C., 144° C., 145° C., 146° C., 147° C., 148° C., 149° C., 150° C., 151° C., 152° C., 153° C., 154° C., 155° C., 156° C., 157° C., 158° C., 159° C., 160° C., 161° C., 162° C., 163° C., 164° C., 165° C., 166° C., 167° C., 168° C., 169° C., 170° C., 171° C., 172° C., 173° C., 174° C., 175° C. or 176° C.

In some methods described herein, the maximum temperature of the preparation during the compressing step is maintained below about 105° C.

In some methods described herein, the maximum temperature of the preparation during the compressing step is maintained below about 150° C.

In some methods described herein, the compressing step is carried out for a duration of from about 1 second to about 120 seconds.

In some methods described herein, the forming comprises a step of compressing the second plurality of plant material fragments at a pressure between approximately 300 and 1500 PSI.

In some embodiments, the storage humidity can be a humidity of plant material, flowable composition, or flowable powder during the compression process. In some embodiments, the storage humidity can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the second ambient relative humidity can be a humidity of the air or environment surrounding the system during compressing. In some embodiments, the second ambient relative humidity can be between 30% RH to 90% RH. In some embodiments, the second ambient relative humidity during compressing can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% humidity.

In some embodiments, the system used for compressing the powder material can be a HUUE Puck Press. In some embodiments, the HUUE Puck Press can process plant material in an environment suitable for compressing. In some embodiments, the HUUE Puck Press can process plant material in an environment with an ambient air temperature not in excess of +65 F and a second ambient relative humidity not in excess of 35%. In some embodiments, the HUUE Puck Press can process plant material in an environment with an ambient air temperature, wherein the ambient air temperature can be OF, 1 F, 2 F, 3 F, 4 F, 5 F, 6 F, 7 F, 8 F, 9 F, 1OF, 11 F, 12 F, 13 F, 14 F, 15 F, 16 F, 17 F, 18 F, 19 F, 20 F, 21 F, 22 F, 23 F, 24 F, 25 F, 26 F, 27 F, 28 F, 29 F, 30 F, 31 F, 32 F, 33 F, 34 F, 35 F, 36 F, 37 F, 38 F, 39 F, 40 F, 41 F, 42 F, 43 F, 44 F, 45 F, 46 F, 47 F, 48 F, 49 F, 50 F, 51 F, 52 F, 53 F, 54 F, 55 F, 56 F, 57 F, 58 F, 59 F, 60 F, 61 F, 62 F, 63 F, 64 F, 65 F, 66 F, 67 F, 68 F, 69 F, 70 F, 71 F, 72 F, 73 F, 74 F, 75 F, 76 F, 77 F, 78 F, 79 F, 80 F, 81 F, 82 F, 83 F, 84 F, 85 F, 86 F, 87 F, 88 F, 89 F, 90 F, 91 F, 92 F, 93 F, 94 F, 95 F, 96 F, 97 F, 98 F, or 99 F. In some embodiments, the HUUE Puck Press can process plant material in an environment where the second ambient humidity can be a % RH, wherein the % RH can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

In some embodiments, the HUUE Puck Press can run in production run. In some embodiments, the HUUE Puck Press can process 1500 pucks per hour. In some embodiments, the HUUE Puck Press can process 9000 pucks per day.

In some embodiments, the flowable composition is formulated. In some embodiments, the plant material is formulated by compressing the flowable composition. In some embodiments, the compressing comprises maintaining the flowable composition at a storage temperature. In some embodiments the storage temperature is between 40 F and 70 F. In some embodiments, the compressing comprises maintaining the flowable composition at a storage humidity. In some embodiments the storage humidity is between 0% and 40%. In some embodiments, the storage temperature is modifiable based on the ambient relative humidity which can be the first ambient relative humidity or second ambient relative humidity. In some embodiments, the storage temperature is increased based on the ambient relative humidity. In some embodiments, the storage temperature is lowered based on the ambient relative humidity. In some embodiments, the ambient relative humidity is the same during the milling and the compressing.

In some embodiments, the first plurality of plant material fragments transferred to a sealable container and are stored in a freezer. In some embodiments, the second plurality of plant material fragments transferred to a sealable container and are stored in a freezer. Sealing the first and/or the second plurality of plant material fragments prevents moisture from coming into contact with the first or second plurality of plant material fragments.

In some embodiments, moisture in the ambient air is reduced. In some embodiments, the moisture is reduced by an air dehumidifier.

In some embodiments, the formulation comprises a puck or a tablet. In some embodiments, the circumference of the puck or tablet can be between Imm and 1 Omm in diameter. In some embodiments, the puck or tablet comprises an aperture therein. In some embodiments the aperture therein is less than the diameter of the puck or tablet. In some embodiments, the puck or tablet comprises an aperture therein. In some embodiments, the puck or tablet can weigh between approximately IOO mg and 200 mg.

In some embodiments, powder to be processed is contained above a feed shoe to a die cavity. In some embodiments, a rotary servo actuator moves the feed shoe over the die opening and ‘shakes’ the feed show (depending on configuration) to fill the die cavity.

In some embodiments, after die-fill, the feed shoe retracts out of the compression area and upper tool tip lowers into the die cavity. In some embodiments, the upper tool lowers and the lower tool raises to compress the powder interposed between the punch tips. In some embodiments, movement of the upper and lower punches may be controlled by servo-actuated motors situated above and below the press frame. In some embodiments, the compression profile can be controlled by software using ‘virtual cams’ to govern the motion and speed of the punches.

In some embodiments, after compression, the upper tool retracts up and out of the die and the lower tool ejects the compressed tablet up and out of the die cavity. In some embodiments, the feed shoe extends and the knock off bar mounted to the face of the feed shoe separates the tablet from the lower punch face and pushes it to the discharge chute. In some embodiments, from this position, the lower tool retracts to the die fill position, the die cavity is filled with powder, and the process may repeat.

In some embodiments, the compression occurs under a pressure not in excess of 7000 PSI (i.e., no more than approximately 3 tons). In some embodiments, the compression occurs under a pressure between 10 PSI and 7000 PSI.

In some embodiments, a user can control lower and upper tools or press components to reach a desired level of compression. In some embodiments, compression occurs in a compression chamber. In some embodiments, the compression chamber is up to 60 mm in depth.

In some embodiments, following the compressing step, the formulation is heated at a temperature above about 105° C. but below about 150° C. for a duration of about 5 minutes to about 30 minutes.

In some embodiments, the compress10n is light or low-end compress10n. In some embodiments, the compression is heavy or high-end compression. In certain cases, the plant material can require more or less compression depending on the profile and/or composition or content of the plant material.

In some embodiments, prior to modifying the plant material, the Cannabis preparation has been heated to a temperature above about 105° C. but below about 150° C. for a duration of about 5 minutes to about 30 minutes. In some embodiments, immediately following the compressing step, the preparation is cooled to a temperature below 10° C. In some embodiments described herein, further comprise packaging the puck or tablet individually in a blister pack impermeable to gas exchange. In some embodiments described herein, further comprise packaging the puck in a re-sealable multi-puck package impermeable to gas exchange.

In some embodiments described herein, the compressing step is performed in a compression mold shaped to provide a signifier embossed on the puck which provides a visual indication of information on the defined dose of selected cannabinoids in the composition, when the process comprises a further step of laser engraving a signifier on the puck which provides a visual indication of information on the defined dose of selected cannabinoids in the composition, or a combination thereof.

In some embodiments described herein, the composition comprises packaging for containing the puck, the method further comprising a step of disposing the composition in packaging configured to associate the puck with a signifier which provides visual information on the defined dose of selected cannabinoids in the composition. In some embodiments described herein, the Cannabis plant material includes material derived from one or more members of a plant variety selected from the group consisting of Cannabis sativa, Cannabis indica, Cannabis ruderalis, and hybrids thereof. In some embodiments described herein, the Cannabis plant variety is Cannabis indica. In some embodiments described herein, the Cannabis plant material is prepared from Cannabis inflorescence. In some embodiments described herein, prior to modifying the plant material further comprises additives selected from among terpenes, terpenoids, puck stabilizers, humectants, vaporization aids, fillers and flavours. In some embodiments described herein, the Cannabis preparation, prior to modifying the plant material has been previously ground to sieve through a mesh of not larger than 1.5 mm in any surface dimension. In some embodiments described herein, further comprise finishing the puck to provide a high gloss surface. In some embodiments described herein, the ground Cannabis preparation includes about 0.1 to about 1.0 grams total mass.

In some embodiments described herein, the puck possesses a degree of friability such that no more than, or exactly, 1% or 0.66% loss results after a friability test as per USP <1216>. In some methods described herein, the puck possesses a degree of friability that meets specifications provided by USP <1216>. In some embodiments described herein, the mesh or sieve has a mesh size of 30, 60, or 120. In some methods described herein, the mesh or sieve has an average opening size of about 0.595 mm, about 0.250 mm, or about 0.125 mm. In some embodiments described herein, the mesh or sieve has a mesh size of 60. In some embodiments described herein, the mesh or sieve has an average opening size of about 0.250 mm. In some embodiments described herein, the composition is formed by compressing its components into a predetermined shape. In some methods described herein, the shape is a puck shape. In some embodiments described herein, the shape is predetermined to be received by a Cannabis vaporizer.

In some embodiments, powder to be processed is contained above a feed shoe in a vacuum insulated container and fed by gravity through a chilled feed shoe to a die cavity. In some embodiments, a rotary servo actuator moves the feed shoe over the die opening and ‘shakes’ the feed shoe (depending on configuration) to fill the die cavity.

In some embodiments, the plant material is maintained at a chilled temperature and/or low humidity during the pressing process, to maintain a flowable state and avoid complications due to material becoming sticky and or viscous at ambient conditions, which may be STP (Imperial units).

### Dosage

In some embodiments, the defined dose Cannabis pucks of the present disclosure have defined doses of one or more physiologically active compounds from Cannabis. In some embodiments, the pucks of the present disclosure have defined doses of one or more cannabinoids. Preferred defined dosages apply to cannabinoid compounds including, but not limited to: THC, THCA, CBD and CBDA. In some embodiments, the pucks may comprise a defined dose selected from the following ranges: about 0 mg, 1 mg, 2, 3, 4, 5, 6, 7, 8, 9, or 1O mg, 20 mg, 30 mg, 40 mg, 50 mg, 60 mg, 70 mg, 80 mg, 90 mg, 100 mg, or any about any 1 mg interval between Omg and 1OOmg THC, about 0 mg, 1 mg, 2, 3, 4, 5, 6, 7, 8, 9, or 1Omg, 20 mg, 30 mg, 40 mg, 50 mg, 60 mg, 70 mg, 80 mg, 90 mg, 100 mg, or any about any 1 mg interval between Omg and 1OOmg THCA THCA, about 0 mg, about 7 mg, about 75 mg, about 1 mg, 2, 3, 4, 5, 6, 7, 8, 9, or 1O mg, 20 mg, 30 mg, 40 mg, 50 mg, 60 mg, 70 mg, 80 mg, 90 mg, 100 mg, or any about any 1 mg interval between Omg and 1OO mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any about any 1 mg interval between Omg and 1OO mg CBDA.

In some embodiments, the pucks comprise about 0 mg, 1 mg, 2, 3, 4, 5, 6, 7, 8, 9, or 1O mg, 20 mg, 30 mg, 40 mg, 50 mg, 60 mg, 70 mg, 80 mg, 90 mg, 100 mg, or any about any 1 mg interval between Omg and 1OO mg of one of the foregoing compounds. In some embodiments, the pucks of the present present disclosure have defined dosages for more than one of the foregoing compounds. For example, in some embodiments, the pucks comprise from about 0 mg-1 mg, or any O. 1 mg interval therebetween THC, about 0 mg, about 9 mg, about 90 mg, about 1 mg, 2, 3, 4, 5, 6, 7, 8, 9, or 1O mg, 20 mg, 30 mg, 40 mg, 50 mg, 60 mg, 70 mg, 80 mg, 90 mg, 100 mg, or any about any 1 mg interval between Omg and 1OO mg, about 0 mg, about 7 mg, about 75 mg, about 1 mg, 2, 3, 4, 5, 6, 7, 8, 9, or 1O mg, 20 mg, 30 mg, 40 mg, 50 mg, 60 mg, 70 mg, 80 mg, 90 mg, 100 mg, or any 1 mg interval between Omg and 1OO mg CBD, and/or about 0 mg, about 7 mg, about 75 mg, about 1 mg, 2, 3, 4, 5, 6, 7, 8, 9, or 10 mg, 20 mg, 30 mg, 40 mg, 50 mg, 60 mg, 70 mg, 80 mg, 90 mg, 100 mg, or any 1 mg interval between Omg and 1OO mg CBDA. In some embodiments, the pucks comprise from about 0 mg, about 9 mg, about 90 mg, about 1 mg, 2, 3, 4, 5, 6, 7, 8, 9, or 1Omg, 20 mg, 30 mg, 40 mg, 50 mg, 60 mg, 70 mg, 80 mg, 90 mg, 100 mg, or any about any 1 mg interval between Omg and 100 mg THC, 0 mg-1 mg THCA, or any 0.1 mg interval therebetween, about 0 mg, about 7 mg, about 75 mg, about 1 mg, 2, 3, 4, 5, 6, 7, 8, 9, or 1O mg, 20 mg, 30 mg, 40 mg, 50 mg, 60 mg, 70 mg, 80 mg, 90 mg, 100 mg, or any about any 1 mg interval between Omg and 1OO mg CBD, and about 0 mg, about 7 mg, about 75 mg, about 1 mg, 2, 3, 4, 5, 6, 7, 8, 9, or 1O mg, 20 mg, 30 mg, 40 mg, 50 mg, 60 mg, 70 mg, 80 mg, 90 mg, 100 mg, or any about any 1 mg interval between Omg and 1OO mg CBDA. In some embodiments, the compositions are substantially free of THC-type cannabinoid compounds. For example, in some embodiments the pucks comprise from about 0 mg-1 mg, or any O.1 mg interval therebetween THC, 0 mg-1 mg THCA, or any 0.1 mg interval therebetween, about 0 mg, about 7 mg, about 75 mg, about 1 mg, 2, 3, 4, 5, 6, 7, 8, 9, or 1O mg, 20 mg, 30 mg, 40 mg, 50 mg, 60 mg, 70 mg, 80 mg, 90 mg, 100 mg, or any about any 1 mg interval between Omg and 1OO mg CBD, and about 0 mg, about 7 mg, about 75 mg, about 1 mg, 2, 3, 4, 5, 6, 7, 8, 9, or 1O mg, 20 mg, 30 mg, 40 mg, 50 mg, 60 mg, 70 mg, 80 mg, 90 mg, 100 mg, or any about any 1 mg interval between Omg and 1OO mg CBD.

### Product and Use of the Product

An embodiment of the present disclosure includes a puck or a tablet produced by a method according to the present disclosure. In some cases, the puck or tablet can have a hole therein. In some embodiments, the hole can be a micro hole, wherein the micro hole has a size such that a gas can pass through the puck (i.e., the puck can breathe).

In some embodiments, an apparatus configured to heat the plant material, or a product or formulation thereof, to a heating temperature can have a receptacle wherein the product or a formulation of the plant material is introduced and/or stored for use. In some cases, the apparatus can heat the plant material. In some cases, the apparatus can be a vape. In some cases, the apparatus can be an electronic pipe. In some cases, the apparatus can be any device for heating a plant material to produce an inhalable smoke or vapor. In some cases, the apparatus can heat the product to a temperature between +315 F-+446 F.

Medicinal applications can be where the formulation is used to ease symptoms caused by certain medical conditions.

Recreational applications can be where the formulation is used for enjoyment rather than health benefits.

In some embodiments, the plant material is introduced to a container configured to heat the plant material to a heating temperature. In some embodiments, formulation is introduced to a container configured to heat the plant material to a heating temperature. In some embodiments, the heating temperature is no less than +315 F. In some embodiments, the heating temperature is no less than +315 F. In some embodiments, the heating temperature is between +315 F and +446 F.

In some embodiments, a product is produced comprising any of the aforementioned embodiments. In some embodiments, the product can be used in an apparatus configured to heat the plant material.

In some embodiments, the apparatus compnses a vape, an electronic pipe, or a device configured to heat the product.

In some embodiments, the product can be used in medicinal applications. In some embodiments, the product can be used in recreational applications.

### System

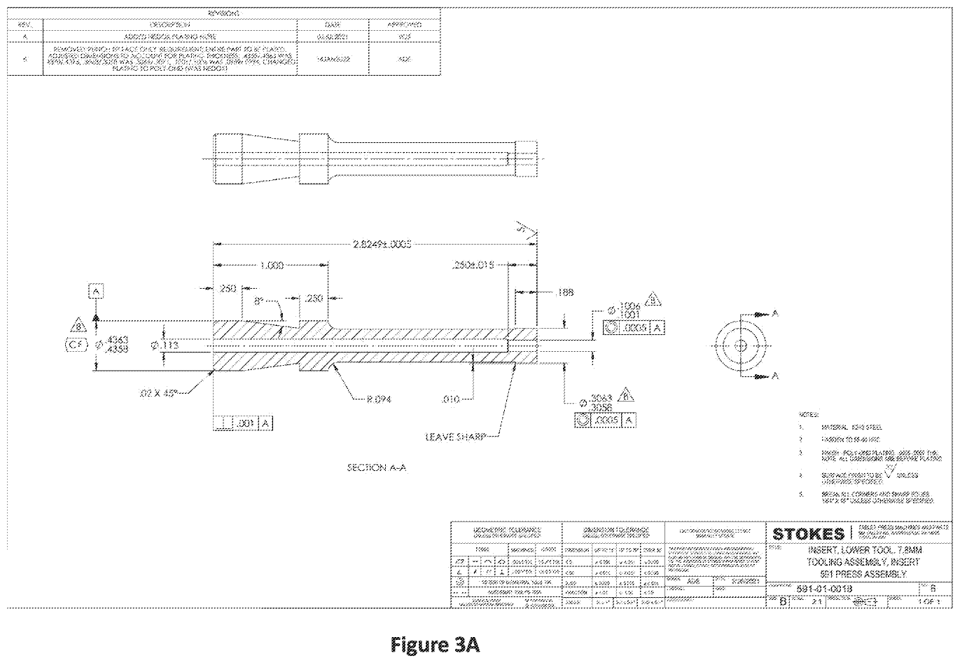

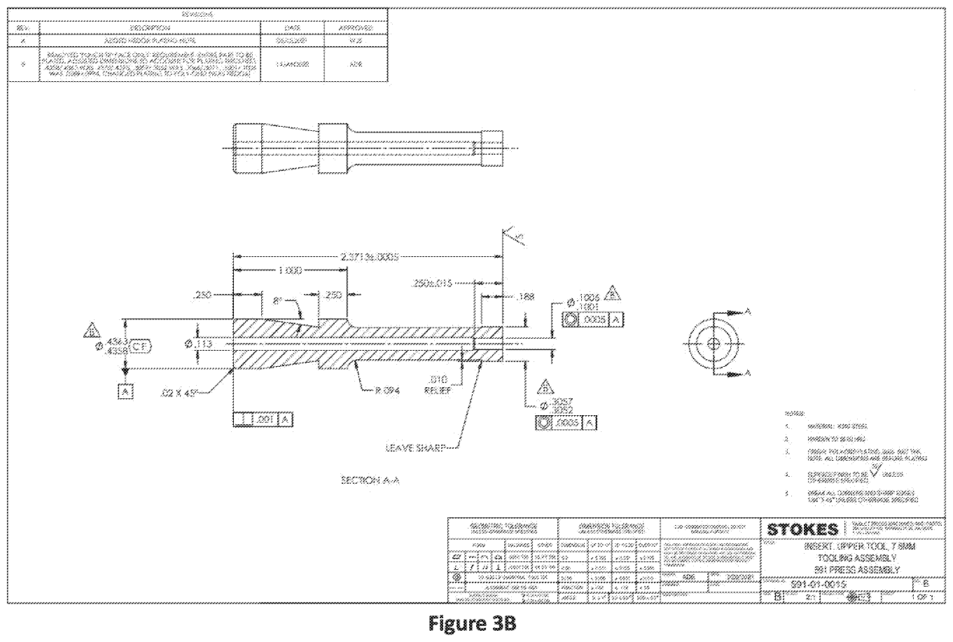

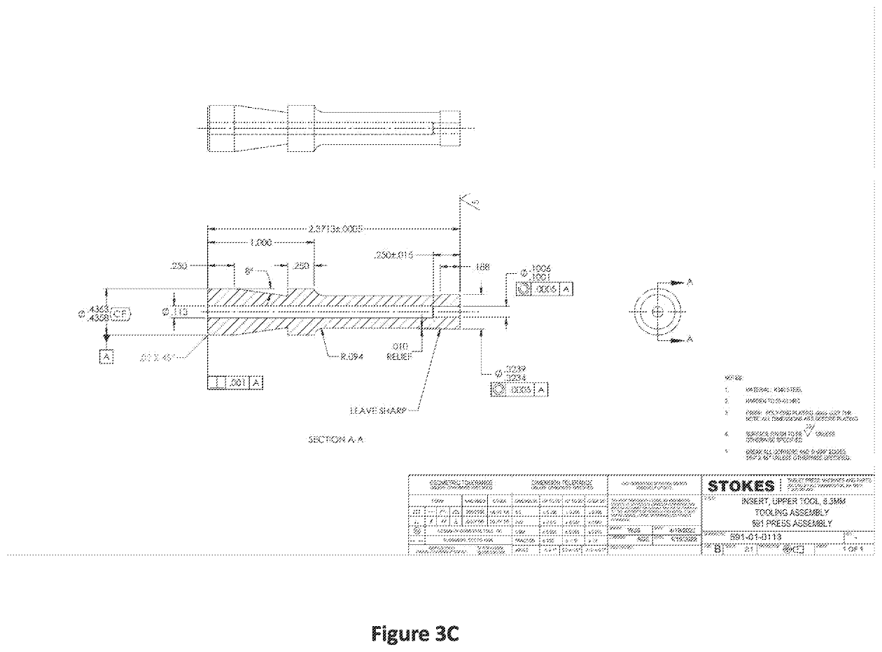

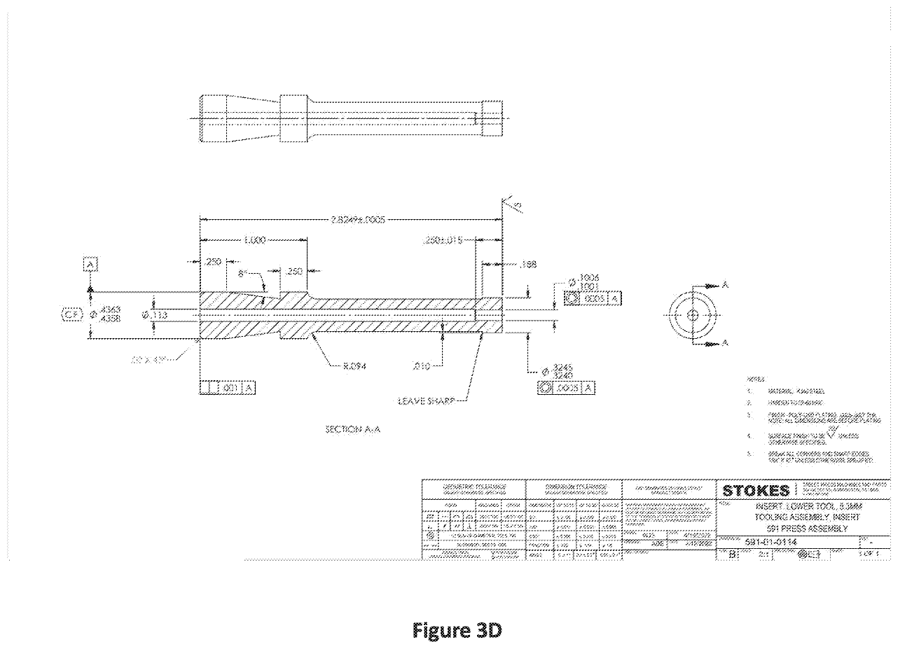

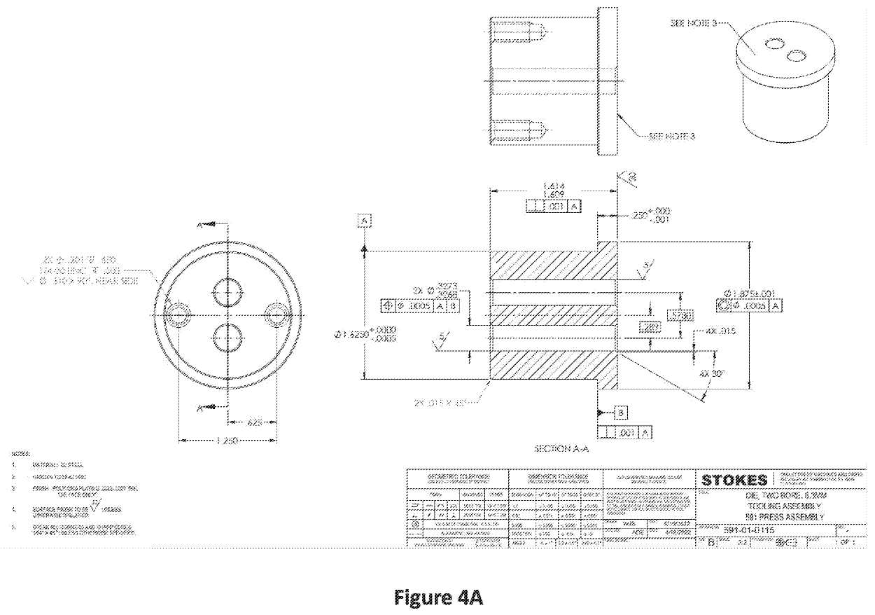

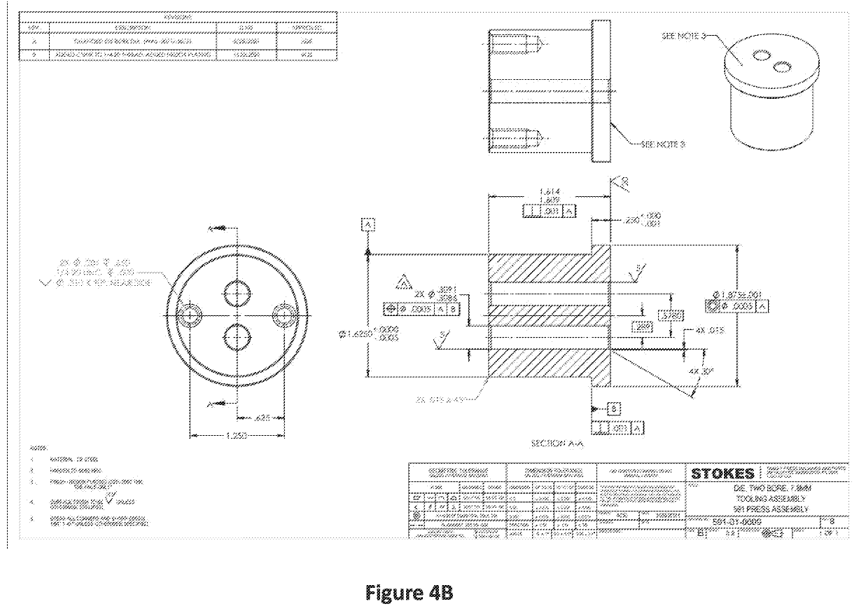

An embodiment of the present disclosure includes a system comprised of a senes of components including a temperature modifying apparatus, a milling device, a dehumidifier, and a compression device. In some embodiments, the temperature modifying apparatus comprises a series of channels. In some embodiments, the series of channels are in direct or indirect contact with each the series of components. In some embodiments, the series of channels transport a coolant to the series of components of the system. In some cases, the coolant can be a liquid or a gas. In some cases, the liquid includes cold water (i.e., no higher than +33 F), antifreeze or a combination thereof. The series of components can be seen in FIGS. 3A-D and FIGS. 4A-B.

The series of channels transporting the coolant are configured to cool each of the series of components. In some embodiments, the coolant moves in a continuous cycle, flowing through each of the series of components and absorbing excess heat produced by the system. In some embodiments, the coolant reduces and removes the heat build-up in the system. In some embodiments, the coolant provides lubrication to reduce friction between parts within each of the series of components, and the system overall. In some embodiments, the coolant flushes away small abrasive particles. In some embodiments, the coolant can protect against corrosion. By altering the mixing ratio or concentration of a coolant, a different balance of cooling and lubrication can result.

In some embodiments, the coolant can be a soluble oil. In some cases, the soluble oil is antimicrobial, antibacterial, antifungal, or a combination thereof. In some embodiments, the coolant can be a synthetic fluid. In some cases, the synthetic fluid contains no mineral content. In some cases, the synthetic fluid is configured to reject unwanted, sump, or contaminant oils. Such unwanted or contaminant oils can be residual oil from the plant material. In some embodiments, the coolant can be a semi-synthetic fluid. These fluids have less oil than emulsion-based fluids and retain much of the same lubricating attributes.

In some embodiments, the coolant is checked to maintain an acceptable concentration level, as concentrations can change 5-20% daily from evaporation, splashing, misting, and drag-out.

In some embodiments, the system comprises a ball mill. The ball mill can be a hard plastic, metal, or combination thereof. In some embodiments, the ball mill can be operated for a length of time that achieves a powdered flower material having a desired average particle size suitable for producing the formulation. In some embodiments, the average particle size is between approximately 300 and 1500 μm. In some embodiments, the length of time is between approximately 1 and 30 minutes. [0144]

In some embodiments, the milling device comprises a tumbling machine comprising a grinder configured to ground the plant material to an average particle size suitable for producing the formulation. In some embodiments, the average particle size suitable for producing the formulation can be between approximately 300 and 1500 μm. in some embodiments, the milling device further comprises an Original Resonator OG CBD Kief Separator, H20 Extraction and Cryo-Trim_{1M} Machine. In some embodiments, the milling device rotates at a rate of between approximately 15 and 150 RPM.

In some embodiments, the compression device can maintain or modify a storage temperature, storage humidity, or combination thereof, of the plant material or first or second plurality of plant material fragments.

### EXAMPLES

These examples are provided for illustrative purposes only and not to limit the scope of the claims provided herein. It will be appreciated that variations in proportions and alternatives in elements of the components shown will be apparent to those skilled in the art and are within the scope of the embodiments presented herein.

### Example 1. Regulation of Temperature and/or Relative Humidity

During the milling and/or pressing steps, depending on operating temperatures and ambient air conditions (namely, temperature and humidity and dew point), water may tend to condense from ambient air onto the systems of the present disclosure. Such condensation can foul the product, reducing its free-flowing properties and preventing a uniform fill into a pressing die, or causing other problems such as damage to the system components via oxidation, or spoliation of plant material. To mitigate this problem, temperature may be controlled based on the ambient relative humidity (RH). As a non-limiting example, to calculate this temperature, a dew point chart is provided below. The vertical columns show air temperature (F), and the horizontal rows show humidity (% RH). The value at the intersection of these rows and columns represents the surface temperature at which condensation will begin to form.

According to Table 1, for example, if the room temperature is 70 degrees F., and the humidity is 55% RH, condensation will begin to form on surfaces below 53 degrees F. If the feed shoe and die are maintained below this temperature (by way of the chiller set point), water will develop on feed shoe and die surfaces. To run at a lower temperature, room temperature may be lowered or air dehumidified to achieve conditions conducive to dry operation.

### Exemplary Press Specifications

Specifications for an exemplary press used in formulating pucks or tablets of the present disclosure are set out in the following table:
Depth of Fill: 38 mm
Maximum Pressing Force: 2 tons
Upper Punch Stroke: 83 mm (18 mm Punch Penetration)
Lower Punch Stroke: 38.2 mm (38 mm Fill Depth, 0.2 mm eject)
Maximum Tablet Diameter: 30 mm (single tip)
Maximum Output: 110 TPM (configuration dependent)
Power Requirements: 480 V, 3 phase, 30 A

### Example 2: Method of Operating the HUUE Puck Press

The plant material is prepared according to the standard methods disclosed in the specification above. The HUUE Puck press may be employed to formulate the flowable powder suitable for pressing into a pressed formulation suitable for vaping applications.

Prior to operating the HUUE Puck Press the operator confirms that it is clean. The operator cleans the HUUE Puck Press if it is not clean. Next, the operator turns the power on to the chiller and the HUUE Puck Press. To ensure that there is enough Glycol, the operator ensures the Glycol levelin the chiller is above the coils. To process up to and greater than 100 grams of material the ambient conditions of the HUUE Puck Press room should be (1) not in excess of 65 F and (2) not in excess of 35% relative humidity. When the ambient relative humidity is in excess of 35%, the amount of plant material in the Thermos container should not be in excess of 100 frams. The operator should place the upper punches of the HUUE Puck Press in chilling position for 15 minutes. Thus, the chilling position is with the punches in the die (compression chamber) to cool the punch tips prior to commencing operations. The operator should set the servos to the HOME position.

When it is time to compress the plant material, the operator should remove a tray out of the freeze dryer and put material into the Thermos Container. The Thermos Container is the custom container placed above the shoe in the Puck Press. The operator should place the Thermos Container into feed shoe at the top back of the Puck Press and close all covers and clear all alarms on the Puck Press.

The operator should run the Puck Press briefly to produce a test puck to set quality and weight. The target weight of the puck is 160 mg, and the puck should be between 140-200 mg.

The compression depends on the plant material strain, the cannabinoid profile, the terpene profile and operating conditions. The objective of the compression is to produce a puck that has “micro holes” having a size such that a gas can pass through the puck (i.e., the puck can breathe), and is also compressed enough so that the puck will maintain its integrity when handled. The compression can be adjusted by increasing or decreasing the fill position or upper punch entrance toggles.

One practical way to accomplish the perfect level of compression is to (1) reduce the compression until you produce the least compressed puck possible, (2) roll the puck back and forth between your fingers without it breaking apart and (3) squeeze the puck until the puck breaks into four pieces instead of collapsing into a powder. The perfect level of compression is referred to as “al dente” (i.e., not over or under compressed).

The HUUE Puck Press can be operated to produce a production run. The HUUE Puck Press should be able to produce about 1500 pucks an hour. The HUUE Puck Press operations include critical, mandatory cleaning breaks/Thus, the HUUE Puck Press can produce about 9000 pucks per day.

The operator should check the compression quality and weight of the produced pucks every 5 minutes and adjust the fill position and/or the upper punch entrance toggles if changes are needed. The operator should perform maintenance on the HUUE Puck Press after every 1000 pucks produced. The operator performs maintenance by removing the upper punch and using a 3/32 drill to remove clogged material and by ensuring that the drill moves freely back and forth thru hole the hole. The operator should use a razor blade to clean the punch tips of material built up, shoe and table residue and wipe the table if necessary. The operator should put the desired amount of material in the freeze dryer for the next day.

The operator should clean the HUUE Puck Press by removing the upper punch, show, lower punch (may be done with less frequency), and Thermos Container. The operator should clean excess buildup from the HUUE Puck Press with a plastic scraper. The operator should put unused and used material in desired places according to regulations such as a vault. The operator should clean all utensils and removable HUUE Puck Press parts in a 3 bay sink with soap & water, rinse, sanitizer. The operator should then clean non-removable parts on the HUUE Puck Press with isopropyl alcohol. The operator should then reassemble the Puck Press.

1. A method of modifying a plant material, the method comprising:
a. providing the plant material, wherein the plant material comprises at least 0.1% v/w of an oil content correlated to a fresh weight associated with a plant flower;
b. milling the plant material at a first temperature to generate a first plurality of plant material fragments, wherein the first temperature is not in excess of +32 F, wherein the first plurality of plant material fragments comprises an average diameter of 300-1500 μm; and
c. exposing the first plurality of plant material fragments to a second temperature not in excess of +32 F and a humidity not in excess of 40% RH for at least 1 hour.

2. The method of claim 1, wherein step c produces a second plurality of plant material fragments.

3. The method of claim 2, wherein the second plurality of plant material fragments comprises a flowable composition.

4. The method of claim 1, wherein the plant material comprises at least 0.13% v/w oil content correlated to fresh weight associated with the plant flower.

5. The method of claim 1, wherein the plant material comprises at least 0.16% v/w oil content correlated to fresh weight associated with the plant flower.

6. The method of claim 1, wherein the plant material comprises at least 0.20% v/w oil content correlated to fresh weight associated with the plant flower.

7. The method of claim 1, wherein the plant material comprises at least 0.29% v/w oil content correlated to fresh weight associated with the plant flower.

8. The method of claim 1, wherein the oil content comprises a monoterpene profile or a sesquiterpene profile.

9. The method of claim 8, wherein the monoterpene profile comprises between approximately 1% and 20% monoterpenes.

10. The method of claim 8, wherein the sesquiterpene profile comprises between 1% and 20% sesquiterpenes.

11. The method of claim 1 or claim 8, wherein the oil content comprises between 1% and 20% composition of monoterpenes.

12. The method of claim 1 or claim 8, wherein the oil content comprises between 1% and 30% composition of sesquiterpenes.

13. The method of claim 1 or claim 8, wherein the oil is a volatile oil or essential oil.

14. The method of claim 1, wherein the plant material comprises between 1% and 30% v/w terpenes correlated to fresh weight associated with the plant flower.

15. The method of claim 14, wherein the terpenes comprise a monoterpene profile or sesquiterpene profile.

16. The method of claim 15, wherein the terpenes comprise between 1% and 30% monoterpenes.

17. The method of claim 15, wherein the terpenes comprise between 1% and 30% sesquiterpenes.

18. The method of claim 15, wherein the plant material comprises between 1% and 30% composition of monoterpenes.

19. The method of claim 15, wherein the plant material comprises between 1% and 30% composition of sesquiterpenes.

20. The method of claim 1, wherein the plant material comprises a cannabinoid profile.

21. The method of claim 1, wherein the second temperature is the same as the first temperature.

22. The method of claim 1, wherein step c occurs after step a and before step b.

23. The method of claim 1, wherein step c occurs after step b.

24. The method of claim 3, wherein the flowable composition comprises particles having an average diameter size between 300 and 1500 μm.

25. The method of claim 1 or claim 3, further comprising producing a formulation comprising the flowable composition.

26. The method of claim 25, wherein producing the formulation further comprises compressing the flowable composition.

27. The method of claim 26, wherein the compression occurs under a pressing force of up to 3 tons.

28. The method of claim 26, wherein the compression occurs under a pressing force between approximately 0.5-1.7 tons.

29. The method of any one of claims 25-28, wherein the formulation comprises a puck or a tablet.

30. The method of claim 29, wherein the puck or the tablet comprises a diameter between approximately 5 mm and 15 mm.

31. The method of claim 29, wherein the puck or the tablet comprises a weight of between 100 mg and 200 mg.

32. The method of claim 29, wherein the puck or tablet comprises an aperture therein.

33. The method of claim 26, wherein the compressing comprises maintaining the flowable composition at a storage temperature, a storage humidity, or a combination thereof, during the compression process.

34. The method of claim 26, wherein the compressing further comprises reducing air moisture via an air dehumidifier.

35. The method of claim 33, wherein the storage temperature is between approximately 30 F and 60 F.

36. The method of claim 33, wherein the storage humidity is not more than 10% RH.

37. The method of claim 1, wherein step a comprises introducing a coolant to an environment containing the plant material, wherein the coolant temperature is not in excess of +32 F.

38. The method of claim 37, wherein the coolant comprises a solid, a gas, or a liquid.

39. The method of claim 38, wherein the gas comprises nitrogen or carbon dioxide.

40. The method of claim 1, wherein the milling in step b) comprises exposing the first plurality of plant material fragments to a first pressure, wherein the first pressure facilitates a shattering or a grinding of the plant material.

41. The method of claim 40, wherein the first pressure is between 1000 and 14,000 kPa.

42. The method of claim 1, wherein the second temperature is not in excess of −50 F.

43. The method of claim 1, wherein the second temperature is between −50 F and +32 F.

44. The method of claim 1, wherein step c is performed for a duration between 1 hour and 16 hours.

45. The method of claim 1, wherein step c results in a reduction of a water content in the plant material by at least 90%.

46. The method of claim 1, wherein step c results in the plant material having a water content not in excess of 10%.

47. The method of claim 1, wherein step c further comprises use of a Harvest Right® Freeze Dryers & At-Home Freeze Drying Accessories I Prep SOS1M.

48. The method of claim 1, wherein the first temperature in step b is modifiable based on a first ambient relative humidity.

49. The method of claim 1, further comprising reducing an ambient temperature of an ambient air, wherein the ambient air has a dew point temperature or a frost point temperature.

50. The method of claim 49, wherein the dew point temperature or frost point temperature of the ambient air is lower than the first temperature, second temperature or storage temperature of the plant material.

51. The method of claim 49, wherein the dew point temperature is no less than +32 F.

52. The method of claim 49, wherein the dew point temperature is between +32 F and +120 F.

53. The method of claim 49, wherein the frost point temperature is not in excess of +32 F.

54. The method of claim 33, wherein the storage temperature is modifiable based on a second ambient relative humidity during the compression process.

55. The method of claim 54, wherein the second ambient relative humidity is the same as the first ambient relative humidity.

56. The method of claim 1, further comprising reducing air moisture via an air dehumidifier.

57. The method of claim 1 or claim 56, wherein reducing air moisture via the air dehumidifier occurs at step c.

58. The method of claim 1, wherein the plant material comprises viscous components, sticky components, or a combination thereof.

59. The method of any one of claims 1-58, further comprising introducing the plant material to a container configured to heat the plant material to a heating temperature.

60. The method of claim 59, wherein the heating temperature is no less than +315 F.

61. The method of claim 60, wherein the heating temperature is between +315 F and +446 F.

62. A product produced by the method of any one of claims 1-61.

63. A use of the product of claim 62 in an apparatus configured to heat the product, the plant material, the formulation or the first or second plurality of plant material fragments.

64. The use of claim 63, wherein the apparatus comprises a vape, an electronic pipe, or device configured to heat the product.

65. A use of the product from claims 63 or 64 in medicinal or recreational applications.

66. A system for producing a flowable particle, the system comprising:
a. a temperature-modifying apparatus configured to alter a temperature of a plant material or a plurality of fragments thereof, wherein the plant material comprises at least 0.1% v/w of an oil content correlated to a fresh weight associated with a plant flower;
b. a milling device configured to process the plant material and produce a plurality of plant material fragments;
c. a dehumidifier configured to alter a moisture content of the plurality of plant material fragments or an ambient relative humidity; and
d. a compression device configured to compress the plant material or the plurality of fragments thereof, wherein the compression produces a formulation.

67. The system of claim 66, wherein the temperature-modifying apparatus comprises a series of channels configured to transport a coolant, wherein the series of channels are located in the milling device and the compression device.

68. The system of claim 67, wherein the coolant comprises a liquid or a gas.

69. The system of claim 66, wherein the milling device further comprises a ball mill.

70. The system of claim 69, wherein the ball mill is a hard plastic, a metal, or a combination thereof.

71. The system of claims 69 or 70, wherein the ball mill is operated for a length of time that achieves a powdered flower material having a desired average particle size to be suitable for producing the formulation.

72. The system of claim 71, wherein the desired average particle size is between 300 μm and 1500 μm.

73. The system of claim 71, wherein the length of time comprises 1 minute to 30 minutes.

74. The system of claim 66, wherein the milling device comprises a tumbling machine comprising a grinder configured to ground the plant material to an average particle size suitable for producing the formulation.

75. The system of claim 75, wherein the average particle size suitable for producing the formulation is between 300 μm and 1500 μm.

76. The system of claim 66, wherein the milling device further comprises an Original Resonator OG CBD Kief Separator, H20 Extraction and Cryo-Trim_{1M} Machine.

77. The system of any one of claims 66-76, wherein the milling device rotates at a rate of 15 RPM to 150 RPM.

78. The system of claim 66, wherein the plant material comprises viscous components, sticky components or a combination thereof.

79. The system of claim 66, wherein the formulation comprises a puck or a tablet.

80. The system of claim 66, wherein the compression device is configured to maintain or modify a storage temperature, a storage humidity, or a combination thereof, of the plant material or plurality of fragments thereof during the compression.

81. The system of claim 66, wherein the oil is an essential oil or a volatile oil.

82. A method of modifying a plant material, the method comprising:
a. providing the plant material, wherein the plant material comprises at most 20% w/w of terpenes correlated to fresh weight;
b. milling the plant material at a first temperature to generate a first plurality of plant material fragments, wherein the first temperature is not in excess of +32 F, wherein the first plurality of plant material fragments comprises an average diameter between approximately 300 μm and 1500 μm; and
c. exposing the first plurality of plant material fragments to a second temperature not in excess of −25 F and a humidity not in excess of 10% RH for at least 1 hour.

83. The method of claim 82, wherein step c occurs after step a and before step b.
